(19) United States
(12) Patent Application Publication
(10) Pub. No.: US 2024/0225734 A1
(43) Pub. Date: Jul. 11, 2024

(54) SYSTEMS AND METHODS FOR DIODE LASER-INDUCED CALCIUM FRACTURES

(71) Applicant: Research Development Foundation, Carson City, NV (US)

(72) Inventors: Thomas E. MILNER, Irvine, CA (US); Marc D. FELDMAN, San Antonio, TX (US); Nitesh KATTA, Irvine, CA (US); Aleksandra GRUSLOVA, San Antonio, TX (US); Drew R. NOLEN, San Antonio, TX (US); Luis A. DIAZ SANMARTIN, San Antonio, TX (US); Scott JENNEY, Irvine, CA (US)

(73) Assignee: Research Development Foundation, Carson City, NV (US)

(21) Appl. No.: 18/408,284

(22) Filed: Jan. 9, 2024

Related U.S. Application Data
(60) Provisional application No. 63/479,123, filed on Jan. 9, 2023.

(51) Int. Cl. A61B 18/24; A61B 18/00; A61B 18/20; A61B 18/22

(52) U.S. Cl. CPC A61B 18/245; A61B 2018/00404; A61B 2018/00732; A61B 2018/00761; A61B 2018/00964; A61B 2018/00982; A61B 2018/2015; A61B 2018/2035; A61B 2018/206; A61B 2018/207; A61B 2018/2211; A61B 2018/2222; A61B 2018/2244; A61B 2018/2261; A61B 2018/2272; A61B 2018/2294

(57) ABSTRACT

Apparatus, systems and methods for fracturing calcium in an artery of a patient. Certain embodiments include a diode laser light source and an optical fiber. In particular embodiments, the optical fiber comprises a polymer or glass optical core, a cladding surrounding the polymer or glass optical core. The optical fiber can comprise one or more emission elements configured to emit electromagnetic energy from the laser light source. The electromagnetic energy can be transmitted through a fluid in the expandable member to fracture the calcium.

### CROSS-REFERENCE TO RELATED APPLICATION

This application claims benefit of priority to U.S. Provisional Patent Application No. 63/479,123 filed Jan. 9, 2023, the entire contents of which is hereby incorporated by reference.

### BACKGROUND INFORMATION

Coronary and peripheral artery calcification (CPAC) complicates percutaneous coronary intervention (PCI) by reducing vessel compliance, interfering with device delivery, impairing balloon expansion, and potentially providing uneven drug distribution in the arterial wall. Further, CPAC causes damage to the drug-eluting polymer, thus resulting in more treatment failures. Decreased vessel compliance also reduces the ability of implanted stents to expand, causing stent failure through under expansion sometimes resulting in complications such as stent thrombosis and restenosis. Calcium localization (superficial or deep), distribution (focal, circumferential or longitudinal extension) and thickness also adversely impacts procedural time and success. CPAC has been an independent predictor of lower survival rate post PCI and is strongly correlated to major adverse cardiovascular events (MACE) after PCI.

Solutions which are currently used clinically to increase vessel compliance and deal with excessive calcium include high pressure balloon inflation, and calcium scoring with cutting balloons. However, these approaches are often unsuccessful because of inability to completely cut calcium, and only applicable to superficial calcium. Coronary atherectomy with both rotational atherectomy systems (e.g., Rotablator™) and orbital atherectomy are suited for removing luminal superficial calcium. However, these approaches do not address deeper calcium and therefore do not always increase vessel compliance sufficiently to assure full stent expansion. These techniques are also technically complex, time consuming and can introduce increased risk since they send cut debris into the micro-circulation which can result in myocardial infarction during the procedure from no-reflow. Thus, dealing with the calcium burden of atherosclerosis in a safe and efficacious manner is a major clinical challenge for interventional cardiologists.

Intravascular lithotripsy (IVL) techniques have been developed using electrical wires, electrodes, and electricity inside a balloon catheter. The electric current discharge creates a vapor bubble formation which during collapse (micro-cavitation) generating sonic pressure waves that travel through soft vascular tissue and can selectively fracture calcium in the vessel wall. The high difference in density and mechanical sonic wave impedance properties between calcium and soft tissue allows the sonic pressure to fracture calcium while leaving soft tissue intact. However, the use of electrical current discharge limits the amount of delivered energy available and the ability to control spatially and temporally the delivery of energy to vaporize the fluid and induce calcium fracture. The electrical current discharge approaches also result in large voltage spikes that can pace the left ventricle with each delivered electric pulse, which is not ideal. There have been multiple cases reported of ventricular fibrillation, and case reports of atrial fibrillation/flutter because the delivered shocks are not timed with the endogenous pacemaker of the heart. Also, the size of the electrodes and wires limit miniaturization of the device. As a result, electric IVL devices are often larger in diameter than the residual lumen of many calcified lesions. As a result, there are many instances where rotational or orbital atherectomy are used simply to deliver the electric IVL device across the calcified lesion. The current electrical current discharge IVL devices are 12 mm in length while most coronary arteries are 100 mm in length. Development of longer balloons for electric IVL would require the addition of more wires and electrodes, further increasing the diameter of the device. Accordingly, systems and methods are desired that overcome these and other limitations associated with existing electrical IVL systems and methods.

### SUMMARY

An urgent need is recognized for the ability to effectively fracture intravascular calcium for treatment of patient conditions, including atherosclerosis and other coronary diseases. Similarly, a need to decalcify heart valves, and the aorta is also recognized. A catheter apparatus is described herein for vascular insertion that employs diode laser sources that can be coupled into small diameter optical fibers to generate cavitation bubbles in a biocompatible fluid that upon collapse generates shock waves that can propagate into the walls of an artery. The optical fibers can be comprised of polymer and/or glass—unlike catheters that use near infrared light that are constructed of a glass material. An important element of our catheter apparatus is the diode laser/biocompatible fluid combination that allows the generation of large pressure-amplitude shock waves (50 atm or more). Indocyanine green is an FDA-approved biocompatible fluid that absorbs strongly at diode laser emission wavelengths (e.g., 700 nm-1000 nm). This particular diode laser/biocompatible fluid combination provides numerous practical advantages. First, the radiant brightness of diode laser sources in the 700 nm-1000 nm spectral range allows the delivery of large pulse energies into small diameter optical fibers (100-400 um) that can be positioned inside arteries with a small lumen diameter. Second, the large pulse energy propagating down the optical fiber does not generate any non-specific electrical/magnetic fields that can interfere with cardiac function. Third, diode lasers that emit light in the 700 nm-1000 nm spectral range can propagate through polymer optical fibers and allow more design flexibility for optical emission elements in the catheter. Fourth, diode laser sources that emit in the 700 nm-1000 nm spectral range can be directly electric-current pulsed to provide light emission allowing precise temporal control over the light emission time and pulse duration. Fifth, because ICG can absorb in the 700-1000 nm spectral range diode laser sources allow incorporation of numerous emission wavelengths that can be coupled into a small diameter optical fiber with multiple optical emission emitting elements. Moreover, the multiple optical emission elements can be each selected for a specific diode laser emitter(s) that can be controlled by electric-current pulsing the appropriate diode emission element(s). More specifically, we disclose specific diode laser emission wavelengths, pulse durations, and formulations of ICG (concentrations and compositions) that when combined together unexpectedly generate large amplitude shock waves that can fracture calcium in the walls of coronary arteries.

Although near infrared pulsed lasers have been applied for decades to generate shock waves in saline due to cavitation bubble generation and/or collapse, these lasers are typically expensive, bulky and can generate shock waves with limited pressure magnitude. Diode laser sources can provide a number of practical advantages in numerous medical therapeutic systems. Diode lasers provide photons at high economic value—the economic cost per radiant watt is less than competing sources and has been decreasing exponentially over the last few decades and is expected to continue decreasing in the future. Generation of shock waves for fracturing calcium in arteries using diode laser sources can provide a number of important advantages. First, the radiant brightness of diode laser sources allows the delivery of large pulse energies into small diameter (100 um-1000 um) optical fibers that can be positioned inside arteries with a small lumen diameter. Second, the large pulse energy propagating down the optical fiber does not generate any non-specific electrical/magnetic fields that can interfere with cardiac function. More specifically, the electric fields associated with large pulse energies are confined to the optical fiber and do not extend outside the catheter and thus do not interfere with surrounding tissues such as the heart. Third, diode lasers emit light that can propagate through polymer and/or glass optical fibers and allow more design flexibility for optical emission elements. Fourth, diode laser sources can be directly electric-current pulsed to provide light emission allowing precise temporal control over the light emission time and pulse duration. Precise temporal control over the light emission allows control over the generation/collapse of generated cavitation bubbles and the corresponding shock wave pressure magnitudes and direction of travel. Fifth, diode laser sources allow selection of numerous emission wavelengths that can be matched to the specific absorption profiles of candidate biocompatible fluids and directed to one of multiple optical emission emitting elements in the fiber catheter that can be selected by electric-current pulsing the appropriate diode emission element.

Previously an apparatus that uses a diode laser source to generate shock waves in a biocompatible fluid and propagate into a biological tissue had not been realized. The biocompatible fluid must be safe when injected into a human coronary artery. Preferably, the biocompatible fluid is already approved by a regulatory agency (e.g., US Food and Drug Agency) for injection into coronary arteries. In previous work, the primary biocompatible fluid contemplated for generating shock waves inside an artery is saline. In existing electrical-source embodiments for generating shock waves, saline is the biocompatible fluid contemplated. In existing light-source embodiments for generating shock waves in arteries, near infrared laser sources that emit in the 1.9-2.1 um spectral range and are strongly absorbed by water have been applied. The use of near infrared laser sources to generate shock waves has a number of limitations. For example, if multiple shock wave emitters are desired, a multiplexing approach is employed where light from one laser source is sequentially spatio-temporally multiplexed into multiple optical fibers. What is needed is a device that can employ economic diode laser sources that can be coupled into a small diameter optical fiber catheter and a biocompatible fluid that when a short pulse of light is injected into the biocompatible fluid a cavitation bubble is generated and collapses creating a shock wave.

Exemplary embodiments of the present disclosure include an apparatus comprising a diode laser light source and an optical fiber, where the optical fiber comprises: a polymer optical core; a cladding surrounding the polymer optical core; and a laser light emission element. In certain embodiments the laser light emission element is a first laser light emission element in a plurality of laser light emission elements. In particular embodiments each of the plurality of laser light emission element is configured to emit light at an equivalent wavelength range. In some embodiments each of the plurality of laser light emission elements is configured to emit light at equivalent power.

In specific embodiments a first laser light emission element of the plurality of laser light emission elements is configured to emit light at a first wavelength range, a second laser light emission element of the plurality of laser light emission elements is configured to emit light at a second wavelength range, and the first wavelength range is different than the second wavelength range. In certain embodiments an optical grating within the optical fiber comprises the plurality of laser light emission elements. In particular embodiments the plurality of laser light emission elements emit light radially from the optical fiber. In some embodiments the plurality of laser light emission elements is configured as a line of scattering centers along the polymer optical core of the optical fiber. In specific embodiments the plurality of laser light emission elements is configured as scattering centers located at positions offset from the polymer optical core and placed at equivalent angles near the cladding. In certain embodiments the plurality of laser light emission elements is configured as one or more photonic crystal lattices comprising a plurality of scatting centers in the polymer optical core.

In particular embodiments the plurality of laser light emission elements comprises N number of laser light emission elements, and wherein laser light emission elements are positioned radially around the optical fiber such that there are 360/N degrees between each laser light emission element in the plurality of laser light emission element. In some embodiments the plurality of laser light emission elements emit light radially 360 degrees around the optical fiber. In specific embodiments the diode laser light source is configured to emit laser light at a wavelength between approximately 690 nanometers (nm) and 900 nm. In certain embodiments the diode laser light source can provide a pulse of light between 50 nanoseconds and 150 microseconds. In particular embodiments radiant power propagating in the optical fiber is between 100 watts (W) and 100 kilowatts (kW). In some embodiments the polymer optical core comprises poly(methyl methacrylate) (PMMA), polydimethylsiloxane (PDMS), polyacrylamide (PAM) or a transparent amorphous fluoropolymer. In specific embodiments the polymer optical core comprises a transparent thermoplastic. In certain embodiments the transparent thermoplastic is poly(methyl methacrylate). In particular embodiments the polymer optical core comprises a silicon-based organic polymer. In some embodiments the silicon-based organic polymer is polydimethylsiloxane. In specific embodiments the polymer optical core comprises a transparent amorphous fluoropolymer. In certain embodiments the polymer optical core comprises a synthetic polymer.

Particular embodiments further comprise an expandable member. In some embodiments the expandable member comprises a lumen configured to receive the optical fiber. In specific embodiments the expandable member contains a fluid. In certain embodiments the fluid surrounds the optical fiber and wherein the fluid absorbs light emitted by the diode laser light source. In particular embodiments the fluid comprises indocyanine green (ICG). In some embodiments the fluid comprises a solvent, and in specific embodiments the concentration of the ICG to the solvent is between 5 milligrams/milliliter (mg/ml) and 25 mg/ml. In certain embodiments the solvent comprises water, saline or dextrose.

Particular embodiments further comprise a control system configured to control an operational parameter of the diode laser light source. In some embodiments the operational parameter is a pulse duration, a wavelength frequency, multiple varying wavelength frequencies, or a wavelength amplitude of the diode laser light source. In specific embodiments the control system is configured to provide a first laser light emission and a second laser light emission from the diode laser light source. In certain embodiments the first laser light emission is configured to generate a bubble in the fluid in the expandable member. In particular embodiments the control system is configured to provide the second laser light emission from the diode laser light source when the bubble in the fluid in the expandable member collapses.

In specific embodiments the optical fiber comprises an imaging element, and in certain embodiments the imaging element is configured to provide intravascular ultrasound (IVUS) or optical coherence tomography (OCT) imaging data. In certain embodiments the diode laser light source is a first diode laser light source in a plurality of diode laser light sources, and the optical fiber is a first optical fiber in a plurality of optical fibers. In particular embodiments each diode laser light source in the plurality of diode laser light sources is coupled to a separate optical fiber in the plurality of optical fibers. In some embodiments an optical fiber in the plurality of optical fibers comprises a conical distal end.

In specific embodiments the plurality of optical fibers are coupled via a tapered fiber coupler. In certain embodiments the plurality of optical fibers are coupled via a side-coupling region. In particular embodiments the plurality of optical fibers are coupled via sleeve coupling elements and at least one of the plurality of optical fibers comprises an angled polished end coated with a dielectric reflector.

Exemplary embodiments include an apparatus comprising: a diode laser light source and an optical fiber, where the optical fiber comprises: an optical core; a cladding surrounding the polymer optical core; and a plurality of laser light emission elements, where the laser light emission elements are configured as emission centers in the optical core. In particular embodiments the plurality of laser light emission elements is configured as a line of scattering centers along the optical core of the optical fiber. In some embodiments the plurality of laser light emission elements is configured as scattering centers located at positions offset from the optical core and placed at equivalent angles near the cladding. In specific embodiments the plurality of laser light emission elements is configured as one or more photonic crystal lattices comprising a plurality of scatting centers in the optical core. In certain embodiments the optical core is a polymer optical core, and in particular embodiments the optical core is a glass optical core.

In specific embodiments the diode laser light source is a first diode laser light source in a plurality of diode laser light sources, and the optical fiber is a first optical fiber in a plurality of optical fibers. In particular embodiments each diode laser light source in the plurality of diode laser light sources is coupled to a separate optical fiber in the plurality of optical fibers. In some embodiments an optical fiber in the plurality of optical fibers comprises a conical distal end.

In specific embodiments the plurality of optical fibers are coupled via a tapered fiber coupler. In certain embodiments the plurality of optical fibers are coupled via a side-coupling region. In particular embodiments the plurality of optical fibers are coupled via sleeve coupling elements and at least one of the plurality of optical fibers comprises an angled polished end coated with a dielectric reflector.

Exemplary embodiments include a method of fracturing calcium in an artery, where the method comprises: inserting an optical fiber into an artery, where the optical fiber is coupled to a diode laser light source, and the optical fiber comprises a polymer optical core, a cladding surrounding the polymer optical core, and a laser light emission element; inserting an expandable member into the artery; expanding the expandable member via a fluid in the expandable member; emitting electromagnetic energy from the laser light emission element, where the electromagnetic energy generates a pressure wave in the fluid contained within the expandable member; and fracturing the calcium in the artery via the pressure wave in the fluid.

In certain embodiments the laser light emission element is a first laser light emission element in a plurality of laser light emission elements. In particular embodiments each of the plurality of laser light emission elements is configured to emit light at an equivalent wavelength range. In some embodiments each of the plurality of laser light emission elements is configured to emit light at equivalent power. In certain embodiments a first laser light emission element of the plurality of laser light emission elements is configured to emit light at a first wavelength range; a second laser light emission element of the plurality of laser light emission elements is configured to emit light at a second wavelength range; and the first wavelength range is different than the second wavelength range. In particular embodiments a grating structure within the optical fiber comprises an element of each laser light emission element. In some embodiments the plurality of laser light emission elements emits light radially from the optical fiber. In specific embodiments the plurality of laser light emission elements comprises N number of laser light emission elements, and wherein laser light emission elements are positioned radially around the optical fiber such that there are 360/N degrees between each laser light emission element in the plurality of laser light emission element.

In certain embodiments the plurality of laser light emission elements emits light radially 360 degrees around the optical fiber. In particular embodiments the diode laser light source is configured to emit laser light at a wavelength between approximately 690 nanometers (nm) and 900 nm. In some embodiments the diode laser light source can provide a pulse of light between 50 nanoseconds and 150 microseconds. In specific embodiments radiant power propagating in the optical fiber is between 100 watts (W) and 100 kilowatts (kW). In particular embodiments the polymer optical core comprises a synthetic polymer. In some embodiments the polymer optical core comprises poly(methyl methacrylate) (PMMA), polydimethylsiloxane (PDMS), polyacrylamide (PAM) or a transparent amorphous fluoropolymer. In specific embodiments the polymer optical core comprises a transparent thermoplastic. In certain embodiments the transparent thermoplastic is poly(methyl methacrylate). In particular embodiments, the optical fiber has a high numerical aperture (NA) so that for a required etendue (or optical throughput) the diameter of the fiber is less. The use of high NA fibers allows the device diameter to be reduced and provides advantages for navigating highly stenotic arteries.

In particular embodiments the polymer optical core comprises a silicon-based organic polymer, and in some embodiments the silicon-based organic polymer is polydimethylsiloxane. In specific embodiments the polymer optical core comprises a transparent amorphous fluoropolymer. In certain embodiments of the method, the fluid comprises indocyanine green (ICG). In particular embodiments the fluid comprises a solvent, and in some embodiments the concentration of the ICG to the solvent is between 5 milligrams/milliliter (mg/ml) and 25 mg/ml. In specific embodiments the solvent comprises water, saline or dextrose.

In certain embodiments the expandable member comprises a lumen, and the optical fiber extends through the lumen of the expandable member. In particular embodiments of the method the optical fiber comprises an imaging element. In some embodiments the imaging element is configured to provide intravascular ultrasound (IVUS) or optical coherence tomography (OCT) imaging. In specific embodiments the imaging element provides imaging data while: inserting the optical fiber into the artery; inserting the expandable member into the artery; expanding the expandable member via a fluid in the expandable member; emitting electromagnetic energy from the laser light emission element; or fracturing the calcium in the artery via the pressure wave in the fluid. In certain embodiments the imaging element provides imaging data after fracturing the calcium in the artery via the pressure wave in the fluid.

In the following disclosure, the term “coupled” is defined as connected, although not necessarily directly, and not necessarily mechanically.

The use of the word “a” or “an” when used in conjunction with the term “comprising” in the claims and/or the specification may mean “one,” but it is also consistent with the meaning of “one or more” or “at least one.” The terms “about” and “approximately” mean, in general, the stated value plus or minus 5%. The use of the term “or” in the claims is used to mean “and/or” unless explicitly indicated to refer to alternatives only or the alternative are mutually exclusive, although the disclosure supports a definition that refers to only alternatives and “and/or.”

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a method or device that “comprises,” “has,” “includes” or “contains” one or more steps or elements, possesses those one or more steps or elements, but is not limited to possessing only those one or more elements. Likewise, a step of a method or an element of a device that “comprises,” “has,” “includes” or “contains” one or more features, possesses those one or more features, but is not limited to possessing only those one or more features. Furthermore, a device or structure that is configured in a certain way is configured in at least that way, but may also be configured in ways that are not listed.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will be apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included to further demonstrate certain aspects of the present disclosure. The invention may be better understood by reference to one of these drawings in combination with the detailed description of specific embodiments presented herein.

FIG. 1 shows a schematic view of an artery with a guidewire for use with an apparatus according to an exemplary embodiment.

FIG. 2 shows a schematic view of an exemplary embodiment according to the present disclosure during an initial stage of use.

FIG. 3 shows a schematic view of a portion of the embodiment of FIG. 1 during use.

FIG. 4 shows a schematic view of a portion of the embodiment of FIG. 1 during use.

FIG. 5 shows schematic end view of an exemplary embodiment according to the present disclosure.

FIG. 6 shows an emission element comprising a beveled surface in an optical fiber.

FIG. 7 shows an emission element comprising an optical grating.

FIG. 8 shows an emission element comprising an embedded optical guide.

FIG. 9 shows emission elements configured as a line of scattering centers along a central region of a core in an optical fiber.

FIG. 10 shows emission elements 150 configured as scattering centers located at positions offset from a core and placed at equivalent angles near a cladding of an optical fiber.

FIG. 11 shows emission elements configured photonic crystal lattice comprising a plurality of scatting centers that are arranged in designed spatial configuration.

FIG. 12 shows a graph of pressure generated according to an exemplary embodiment according to the present disclosure.

FIG. 13 shows a graph of molar extinction coefficient versus wavelength according to an exemplary embodiment according to the present disclosure.

FIGS. 14-18 show graphs of molar extinction coefficient versus wavelength for different fluid combinations and concentrations of comprising indocyanine green ([ICG] according to exemplary embodiments of the present disclosure.

FIGS. 19-23 illustrate data obtained in ICG precipitation tests for different fluid combinations and concentrations of comprising indocyanine green ([ICG].

FIGS. 24-29 illustrate data from emitting a second pulse of electromagnetic energy and timed to occur at the collapse of a vapor bubble generated by a first pulse of electromagnetic energy.

FIG. 30 illustrates a block diagram of a system utilizing multiple diode lasers combined into a single fiber and then split into different fibers for each emitter.

FIG. 31 illustrates a block diagram of a system using a separate small core high NA fiber coupled to each diode and emitter.

FIG. 32 illustrates an end section view of one embodiment of an IVL catheter comprising a plurality of optical fibers distributed around a central guide wire.

FIG. 33 illustrates optical fibers configured to redirect laser light emissions.

FIG. 34 illustrates an end section view of one embodiment of an IVL catheter comprising a plurality of optical fibers distributed around a central guide wire with an aperture in outer coil.

FIG. 35 illustrates schematic section views of an IVL catheter comprising a plurality of optical fibers within an expandable member.

FIG. 36 illustrates a partial section view of an embodiment of a tapered fiber coupler configured for use in an IVL catheter.

FIG. 37 illustrates a partial section view of an embodiment of a side-coupled fiber configured for use in an IVL catheter.

FIG. 38 illustrates a partial section view of an embodiment of inline reflectors created from dielectric film configured for use in an IVL catheter.

### DETAILED DESCRIPTION OF ILLUSTRATIVE EMBODIMENTS

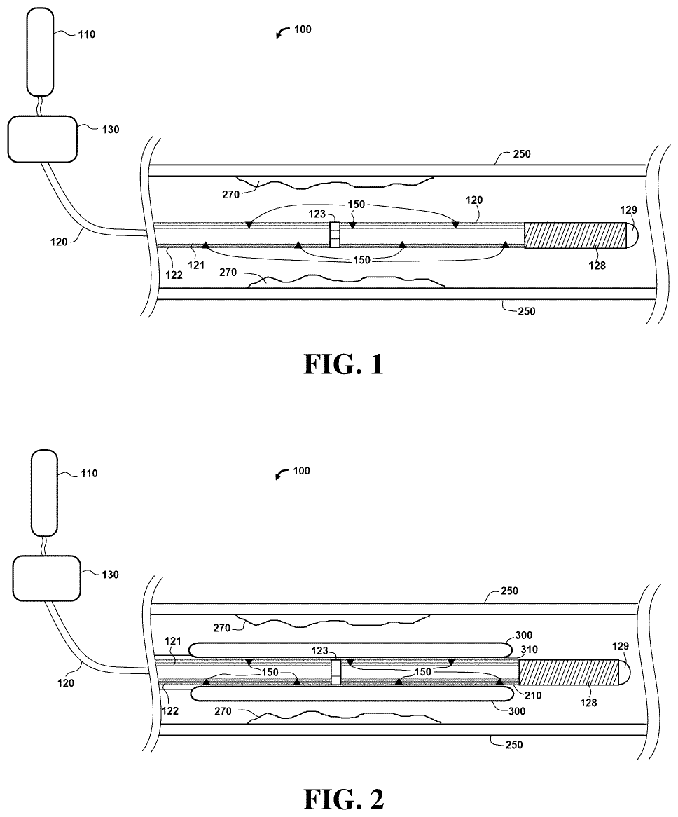

Exemplary embodiments of the present disclosure include apparatus and methods for fracturing arterial calcium, including for example calcium in a coronary artery. Referring initially to FIGS. 1-4, an overview of an exemplary apparatus 100 and method of use are demonstrated. For purposes of clarity, not all features shown in each figure are labeled with reference numbers. In the embodiment shown, apparatus 100 comprises a diode laser light source 110 coupled to an optical fiber 120 and a control system 130. Control system 130 can be configured to control operational parameters of apparatus 100, including for example, the operation of diode laser light source 110 (e.g., laser pulse duration, frequency, amplitude et al.) during calcium fracturing procedures.

In FIG. 1, optical fiber 120 of apparatus 100 has been inserted into an artery 250 with calcium 270 located within artery 250. In FIGS. 1-4, artery 250 and the portion of optical fiber 120 are shown in a cross-sectional view. As will be discussed more fully below, optical fiber 120 comprises an optical core 121 and a polymer cladding 122 surrounding optical core 121. In addition, optical fiber 120 comprises one or more laser light emission elements 150. In exemplary embodiments of the present disclosure, optical fiber 120 is positioned within artery 250 such that laser light emission elements 150 are proximal to calcium 270 (e.g., optical fiber 120 is inserted into artery 250 a sufficient distance until laser light emission elements 150 are generally aligned with calcium 270). In particular embodiments, optical fiber 120 may comprise an imaging element 123 to assist in the positioning of optical fiber 120. In specific embodiments, imaging element 123 may be configured to provide intravascular ultrasound (IVUS) and/or optical coherence tomography (OCT) imaging. In certain embodiments, polymer cladding 122 may comprise poly(methyl methacrylate) (PMMA), polydimethylsiloxane (PDMS), polyacrylamide (PAM) or a transparent amorphous fluoropolymer (e.g. Cytop™). It is understood that other embodiments of the present disclosure may comprise a polymer cladding with a different polymer than those listed herein.

In FIG. 2 an expandable member 300 (e.g. a balloon catheter) has been inserted into artery 250. In certain embodiments, optical fiber 120 can function in a manner equivalent to a typical guidewire to allow for the insertion of expandable member 300. For example, in the embodiment shown, expandable member 300 comprises a lumen 310 configured to receive optical fiber 120 so that optical fiber 120 can be used to guide expandable member 300 into the desired location within artery 250. In addition, optical fiber 120 comprises a distal end 129 with a formable or shapeable portion 128. In particular embodiments, shapeable portion 128 may be formed from a nickel titanium alloy (e.g. Nitinol) wire or other suitable configurations, including for example, workhorse tips available from Asahi©. In exemplary embodiments distal end 129 can be coupled to optical fiber 120 via polyethylene micro tubing or other suitable structures. Existing imaging techniques can also be used to assist in the placement of expandable member 300. In particular embodiments, imaging element 123 can be used to assist in the positioning of expandable member 300 with respect to optical fiber 120 and calcium 270. Accordingly, expandable member 300 can be positioned within artery 250 in a manner that is familiar to surgeons to allow for accurate placement in the desired location proximal to calcium 270.

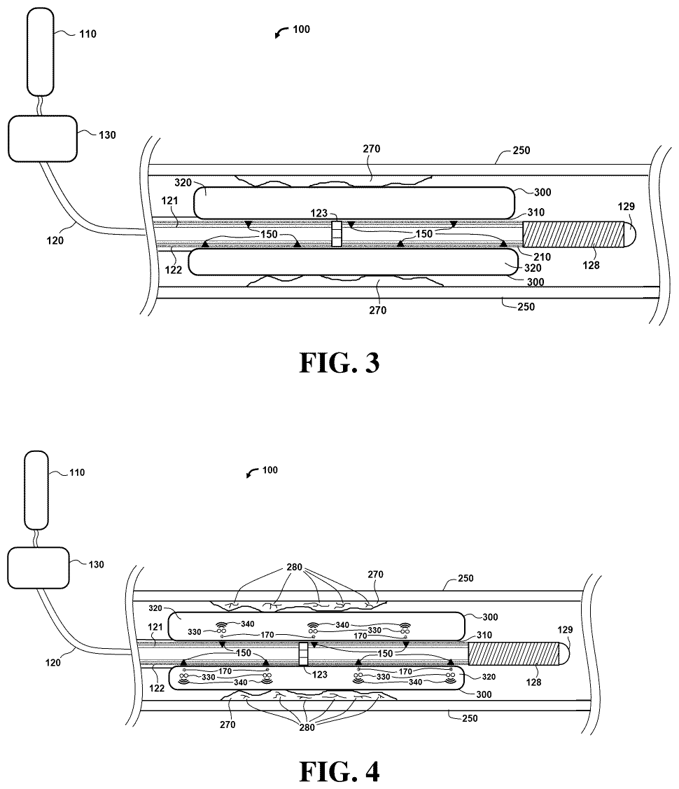

In the embodiment shown in FIG. 3, expandable member 300 has been expanded within artery 250 via a fluid 320 including, for example, a fluid comprising indocyanine green (ICG). In particular embodiments fluid 320 may comprise ICG and a solvent. In certain embodiments, the solvent may comprise a mixture of one or more of saline, dextrose and/or water. Expandable member 300 can be expanded by increasing the pressure of fluid 320 within expandable member 300.

In the embodiment shown in FIG. 3, expandable member 300 has been expanded after expandable member 300 has been inserted into artery 250 (as shown in FIG. 2) and prior to emitting electromagnetic energy 170 from laser light emission elements 150 (as shown in FIG. 4). Electromagnetic energy 170 creates cavitation (e.g. bubbles 330) in fluid 320 which generates ultrasonic waves 340 from the formation and collapse of the bubbles 330 in fluid 320. In certain embodiments a grating structure within optical fiber 120 comprises laser light emission elements 150. In certain embodiments, expandable member 300 can be configured as a balloon configured for treatment of the distal aorta in order to increase compliance of the aorta in elderly patients with resistant systolic hypertension, and to increase elastic recoil during diastole to improve blood flow to the microcirculation.

As shown in FIG. 4, ultrasonic waves 340 propagate through fluid 320 and create fractures 280 only in calcium 270 without damaging the vessel walls of artery 250, since the vessel walls are more elastic than the calcium plaque. In exemplary embodiments, fractures 280 are created along inhomogeneities in calcium 270 and/or in calcium-hard-soft tissue interfaces. Fracturing of calcium 270 increases compliance of artery 250, allowing artery 250 to more easily expand and contract with changes in pressure. In specific embodiments, imaging element 123 can be used to monitor the fracturing of calcium 270.

In particular embodiments elements of apparatus 100 are specifically selected to increase the ability to create fractures 280 in calcium 270 with reduced power requirements from diode laser light source 110 and reduced manufacturing costs for apparatus 100. For example, the laser light source 110 and fluid 320 can each be selected to maximize the amount of energy provided by ultrasonic waves 340 while minimizing the power requirements from diode laser light source 110. In particular embodiments, the operational parameters of laser light source 110 (e.g. the wavelength, pulse duration, etc. of electromagnetic energy 170) and the concentration of ICG in fluid 320 can be selected to optimize the efficiency of apparatus 100 (e.g. the ability to create fractures 280 in calcium 270 for a given power requirement of laser light source 110).

Furthermore, in particular embodiments optical fiber 120 may be formed from fibers with significantly lower costs than glass fibers. In specific embodiments, optical fiber 120 may be formed from fiber material that costs approximately $0.10 per meter, significantly reducing the manufacturing costs for apparatus 100.

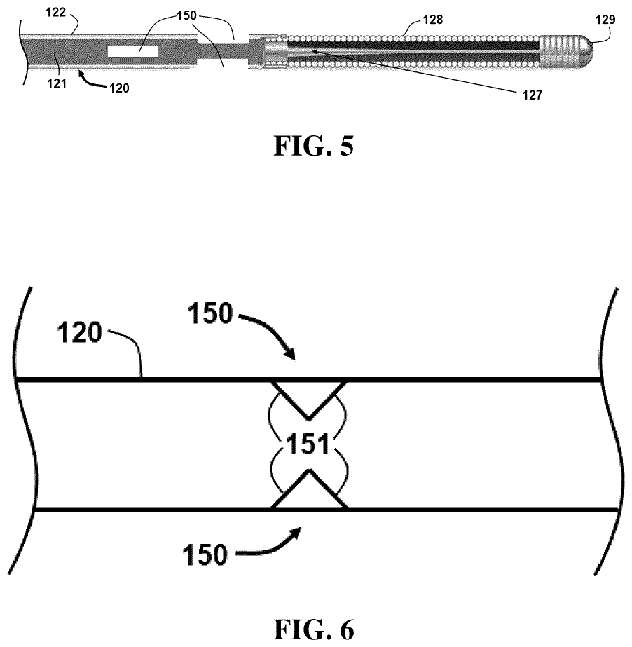

A close-up view of one embodiment of distal end 129 is shown in the partial section schematic view of FIG. 5. As shown in the figure, shapeable portion 128 comprises a tapering core material 127. In certain embodiments, tapering core material 127 may be formed from a metal alloy of nickel and titanium (e.g. Nitinol). In addition, optical fiber 120 is shown with emission elements 150 that are radially arranged at both 90 degrees and 180 degrees from each other to provide for emission of electromagnetic energy around the circumference of optical fiber 120. It is understood that the configuration of emission elements shown in the figures are exemplary and other configurations of emission elements may be utilized according to embodiments of the present disclosure. For example, emission elements 150 may be arranged at 60, 45, 30 degrees (or other configurations as desired) around the circumference of optical fiber 120. In certain embodiments, emission elements 150 may be arranged to emit electromagnetic energy 360 degrees around the circumference of optical fiber 120.

Exemplary embodiments of the present disclosure may comprise emission elements 150 of one or more configurations. FIG. 6 illustrates a closer view of emission elements 150 illustrated in FIGS. 1-4. In the embodiment shown in FIG. 6, emission elements 150 comprise one or more beveled surfaces 151 in optical fiber 120. In certain embodiments, a radial emitting fiber comprises a single wedged surface that acts as both a reflector and refractive element as shown in FIG. 6. Wedged surfaces can be concatenated along the length of the optical fiber to realize multiple emitting elements.

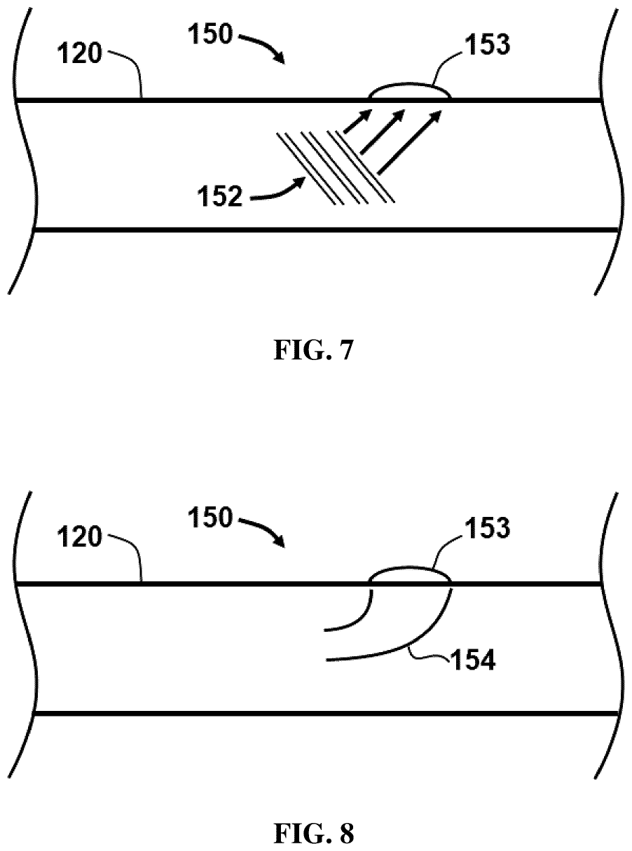

In the embodiment shown in FIG. 7, optical fiber 120 includes emission element 150 comprising a fiber grating 152 and an optional lens element 153. In the embodiment shown in FIG. 8, emission element 150 comprises an optical guide 154 and an optical lens element 153. In exemplary embodiments, optical guide 154 has a refractive index that is higher than optical fiber 120. In certain embodiments, optical guide 154 may be configured as an orthogonal waveguide. Optional lens 153 in FIGS. 7 and 8 can focus electromagnetic energy into biocompatible fluid in an expandable member surrounding optical fiber 120 (e.g. electromagnetic energy 170 into fluid 320 in expandable member 300 shown in FIG. 4). In certain embodiments, dielectric gratings may be written into fibers that are designed to couple light of selected wavelengths out of the fiber and into the surrounding biocompatible absorbing fluid. Waveguides can be written into a fiber to couple out radiation from the core by creating a region of higher refractive index. A waveguide region of higher refractive index can be created by first removing material from the fiber using a subtractive manufacturing process and then filling with a higher-index synthetic polymer.

In exemplary embodiments of the present disclosure, directing light from the optical fiber into the surrounding biocompatible absorbing fluid is accomplished using one or more emission elements configured as optical emitter(s) embedded into the optical fiber. Exemplary embodiments of optical emitter elements comprise a patterned refractive index gradient within the fiber-core guiding structure of the optical fiber. In particular embodiments, the function of the optical emitter elements is to couple to and direct light out of the fiber-core guiding structure and into the surrounding biocompatible absorbing fluid. The patterned refractive index gradient embedded within the fiber-core guiding structure can be of multiple forms and may comprise one or more of: (1) reflective surface; (2) refractive surface; (3) scattering center; (4) dielectric grating; (5) waveguide within the core; and/or (6) photonic crystal lattice.

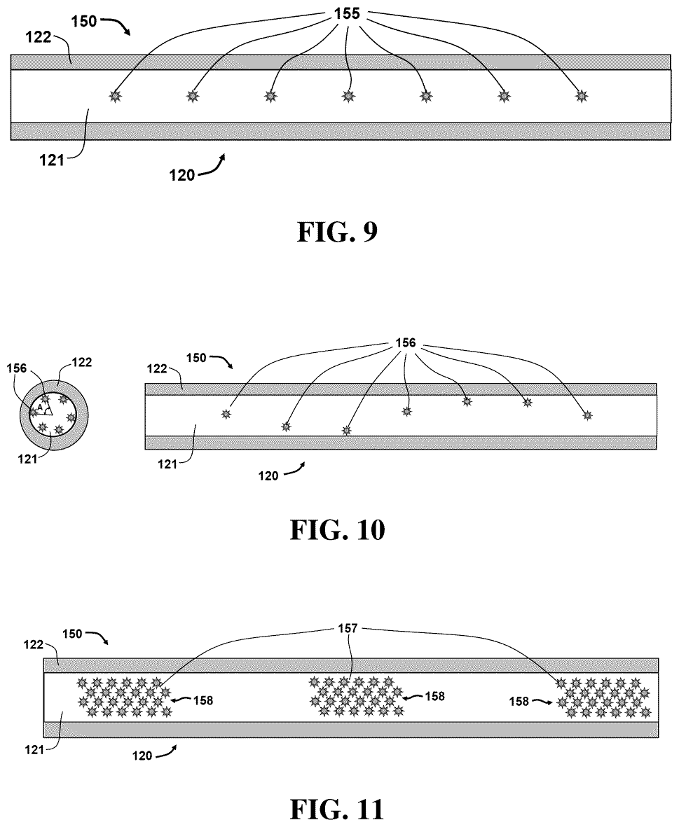

In particular embodiments, the refractive index of selected regions in the core of an optical fiber can be modified (increased or decreased) by directing focused radiation into the core of the optical fiber to create a scattering center. The scattering center can have higher or lower refractive index compared to the surrounding core in specific embodiments. Referring now to FIG. 9, one exemplary embodiment comprises optical fiber 120 with a cladding 122 surrounding an optical core 121 with emission elements 150 configured as a line of scattering centers 155 along the central region of core 121 of fiber 120.

In the embodiment shown in FIG. 10, optical fiber 120 comprises cladding 122 surrounding an optical core 121 with emission elements 150 configured as scattering centers 156 located at positions offset from core 121 and placed at equivalent angles near cladding 122. As shown in the end section view on the left side of FIG. 10, in this embodiment six scattering centers 156 are equally spaced around the peripheral region of core 121 such that an angle A between the adjacent scattering centers 156 is approximately 60 degrees. While the embodiment shown in FIG. 10 comprises six radial locations scattering centers 156 arranged in a spiral or helix pattern along optical fiber 120, it is understood that other embodiments may comprise a different number of radial positions and/or a different arrangement along the length of optical fiber 120. For example, certain embodiments could comprise a different number of radial positions arranged linearly along the peripheral region of core 121.

In particular embodiments, a photonic crystal lattice can be written into the core region of the optical fiber. Referring now to FIG. 11, emission elements 150 are configured as one or more photonic crystal lattice(s) 157 comprising a plurality of scatting centers 158 that are arranged in designed spatial configuration. A photonic crystal lattice 157 may be produced, for example, by a focused laser beam to induce a localized phase transition or scattering center in the core of the optical fiber to create a region of modified refractive index. By scanning the beam focus laterally and/or longitudinally, photonic crystal lattices 157 may be created at discrete longitudinal locations along the fiber core. For any of the patterned refractive index gradients embedded within the fiber-core guiding structure a curved refractive surface may be fabricated onto the fiber or guidewire surface to focus light coupled out of the core into the surrounding biocompatible absorbing fluid.

In addition, the absorbing biocompatible fluid in the expandable member can be configured to efficiently fracture calcium with respect to the electromagnetic energy provided. As molar concentration of ICG increases in solution, the absorption coefficient also increases. However, this increase is not linear. Hence, if 1× concentration is 1 cm⁻¹, 100× is not necessarily 100 cm⁻¹. This is because of an “aggregation” effect of cyanine dyes. Cyanine dyes, including ICG, tend to aggregate at high concentration in aqueous solutions, which can reduce the absorption coefficient.

A lower aggregation implies lower power needed to generate the same pressure. While dimethyl sulfoxide (DMSO) can be used to avoid aggregation in ex vivo applications, it is not biocompatible. Accordingly exemplary embodiments of the present disclosure can comprise other techniques, including for example, dissolving the dye in liposome-type nano droplets. In addition, exemplary embodiments of the present disclosure can utilize dextrose, plasma, albumin and/or water in the solution to increase the absorption coefficient.

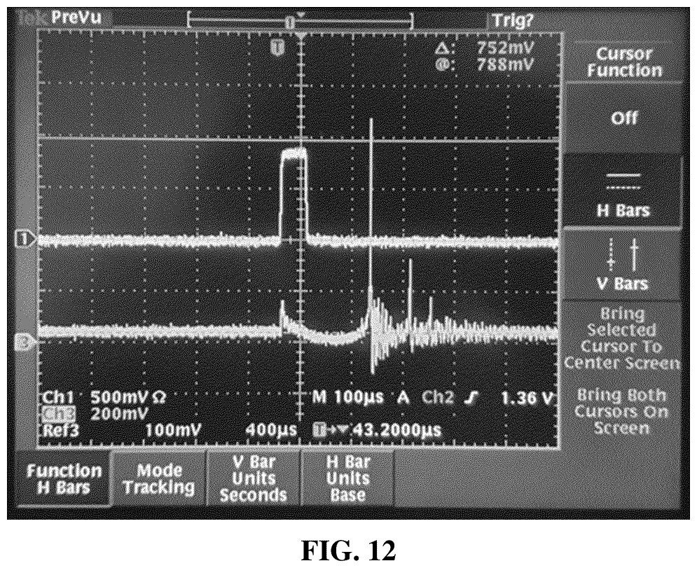

Data from one particular embodiment is shown in FIG. 12. In this embodiment diode laser light source 110 is configured to emit electromagnetic energy 170 at a wavelength of 787 nanometers (with a small spectral bandwidth of less than 5 nanometers [nm]) with a pulse duration of approximately 50 s. In the embodiment shown, the pulse energy is less than 15 millijoule (mJ). When directed into a fluid containing ICG (and optionally a solvent, including for example, water, saline or dextrose) at ICG concentration of 25 milligrams/milliliter (mg/ml), the ultrasonic waves 340 generated a pressure of greater than 50 bars in the fluid.

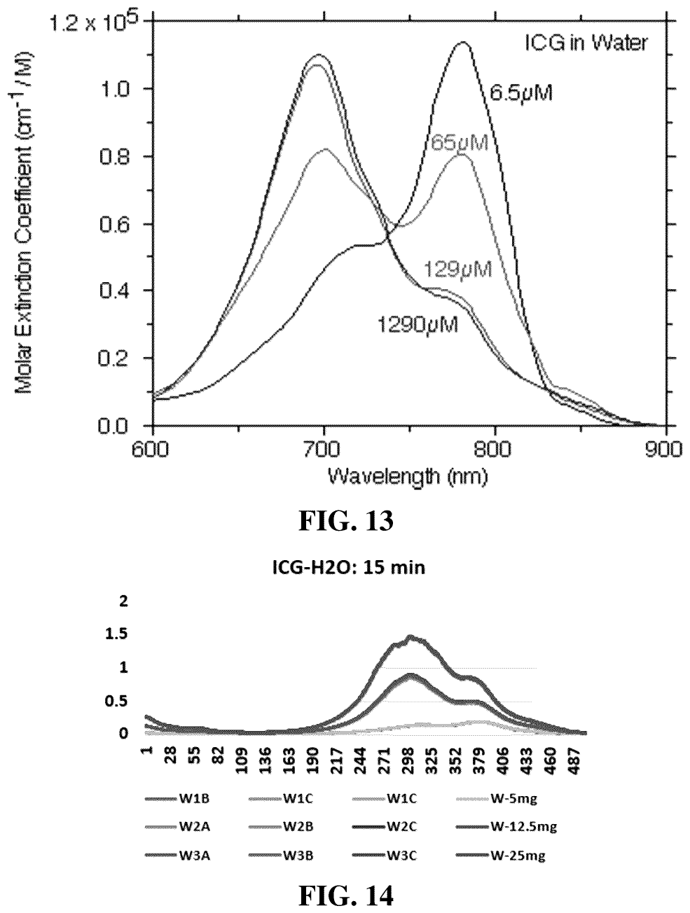

FIG. 13 shows a graph indicating molar extinction coefficient (a measure of how strongly a chemical species or substance absorbs light at a particular wavelength) versus wavelength according for different concentrations of ICG in water. The absorption at the laser wavelength (e.g., approximately 787 nm) is more than 10 times greater than the absorption of water at a laser wavelength of approximately 2 m. In particular embodiments, diode laser light source 110 can be configured to emit electromagnetic energy at a wavelength near the maximum absorption coefficient for a specified concentration of an ICG formulation in expandable member 300. The use of diode lasers also provides for a compact configuration and flexible pulse profile. Accordingly, embodiments utilizing diode lasers can provide sufficient electromagnetic energy to an absorbing biocompatible fluid in an expandable member to effectively fracture calcium.

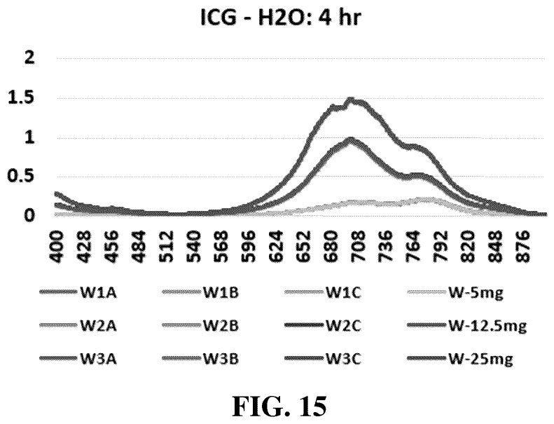

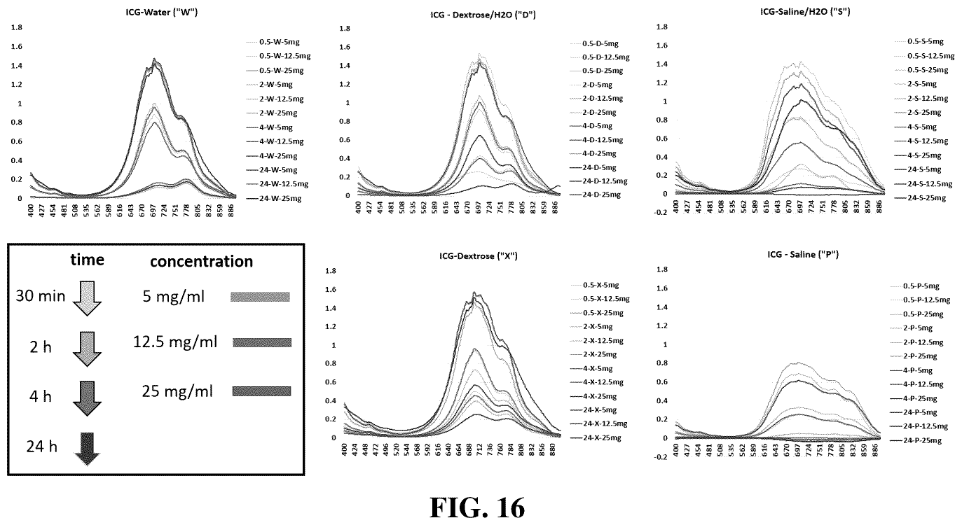

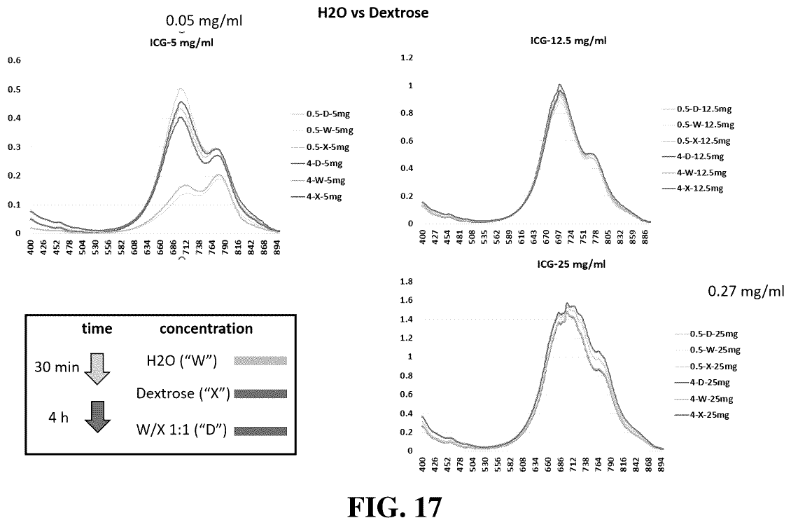

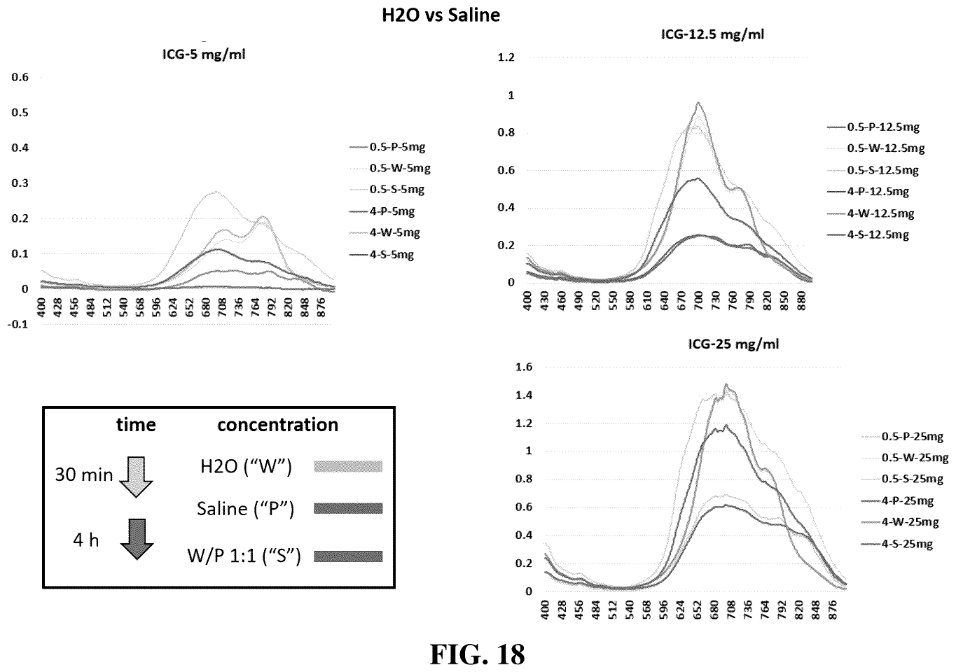

As previously noted, the contents of fluid 320 can be optimized efficiently fracture calcium with respect to the electromagnetic energy provided. In FIGS. 14-18, data was obtained regarding the molar extinction coefficient versus wavelength according for five different ICG solvent combinations at different concentrations using a BioDrop® μLITE+ spectrophotometer from BioChrom®. Specifically, the solvents included water, saline, water/saline (1:1), water/dextrose (1:1) and dextrose at ICG concentrations of 5, 12.5 and 25 mg/ml. As shown in the graphs, dextrose alone or in combination with water does not change the absorption peaks of ICG at concentrations of 5, 12.5 and 25 mg/ml.

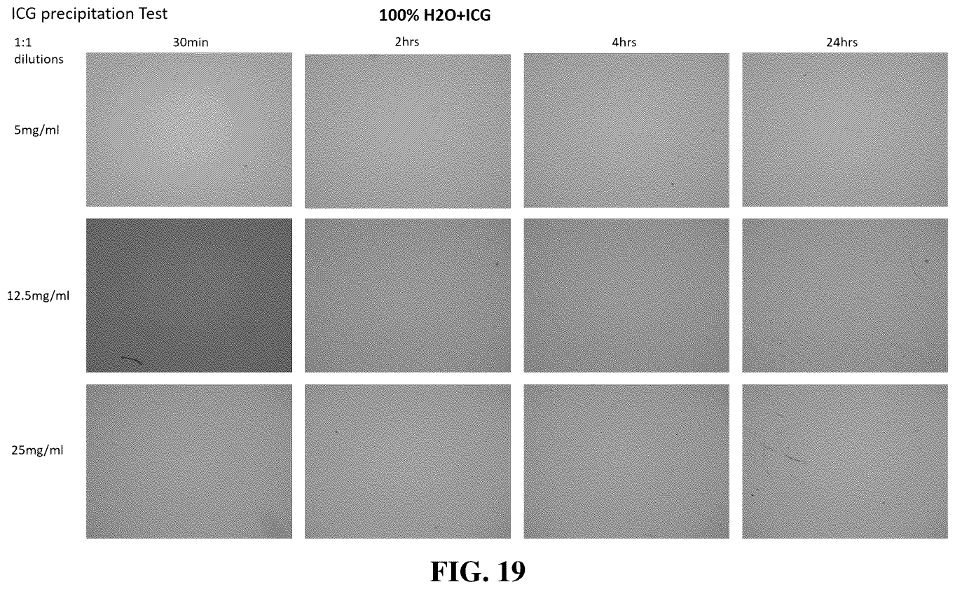

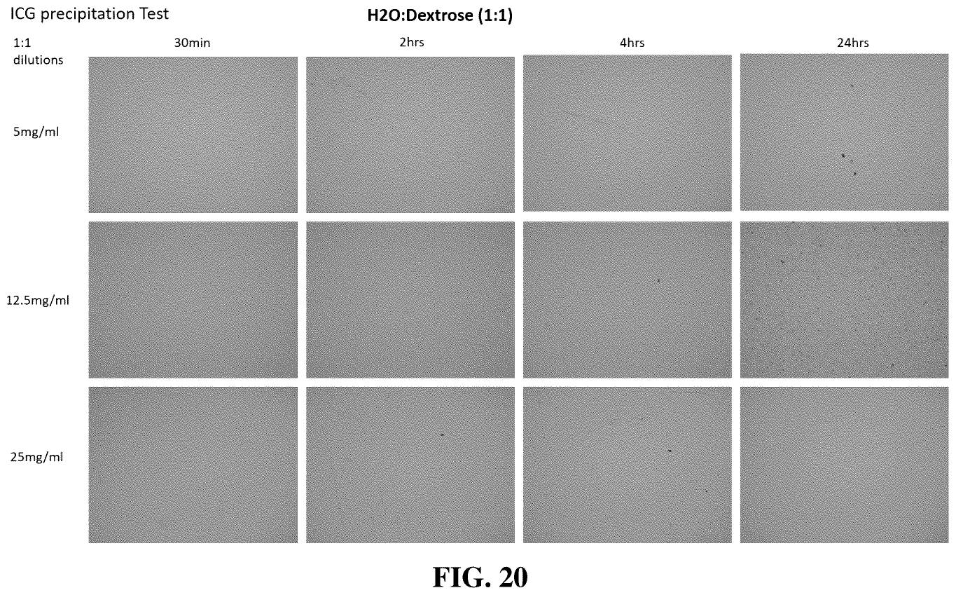

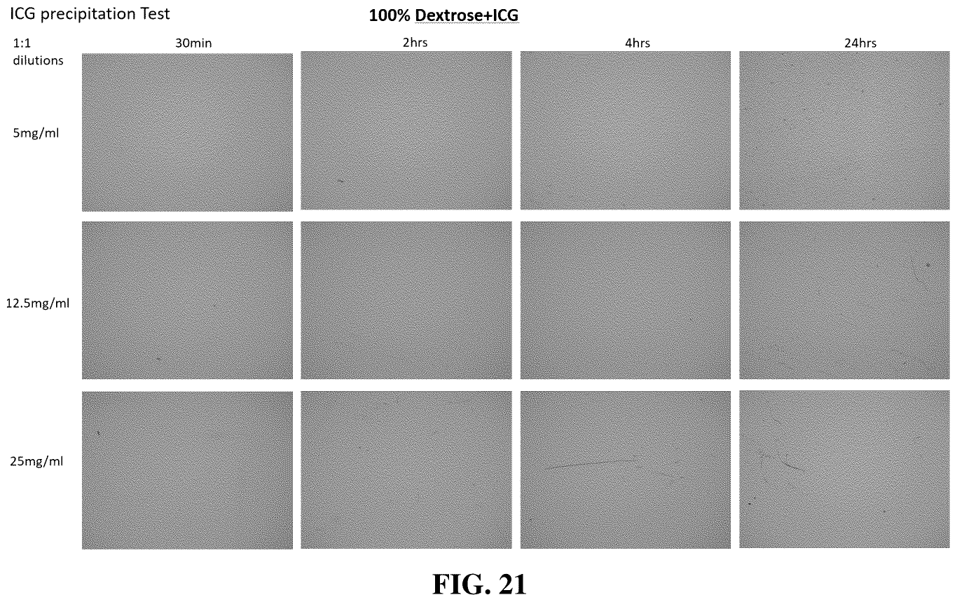

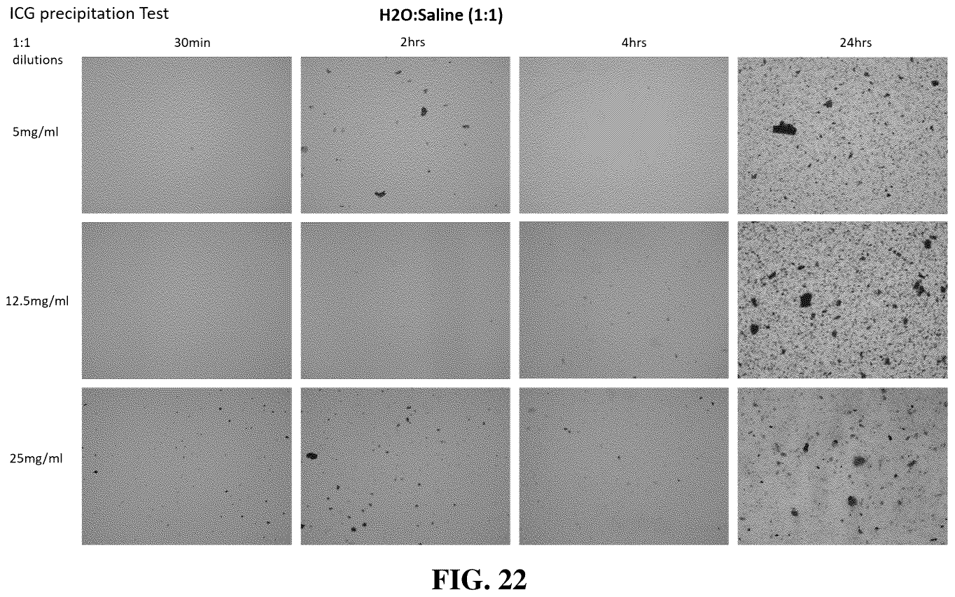

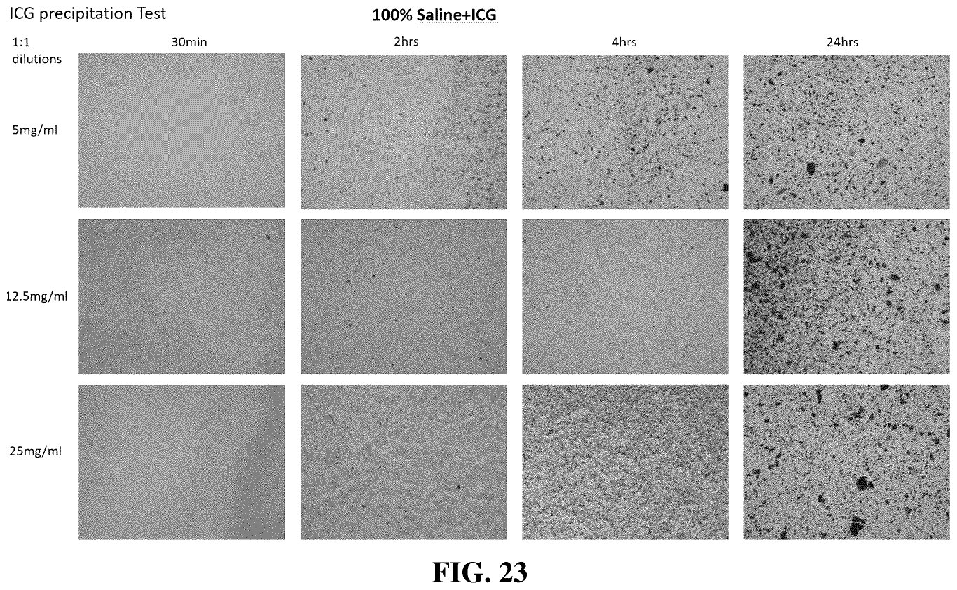

Another factor for consideration when determining a desired ICG formulation is the extent to which ICG precipitates out of solution. FIGS. 19-23 illustrate data obtained in ICG precipitation tests for five different solvents at three different concentrations over time. Again, the solvents used to obtain the data in FIGS. 19-23 included water, saline, water/saline (1:1), water/dextrose (1:1) and dextrose, respectively. The precipitation data in FIGS. 19-23 was obtained with concentrations of ICG in the solvents at 5 mg/ml, 12.5 mg ml and 25 mg/ml at 37° Celsius at 15 minutes, 2 hours, 4 hours and 24 hours using an Invitrogen™ Countess™ II cell counter. The data in FIGS. 19-23 indicate that dextrose alone or in combination with water can be used to reconstitute ICG without causing precipitation within five hours of mixing.

Exemplary embodiments of the present disclosure may also be configured to provide sequential electromagnetic energy (e.g. laser light) emissions specifically time to maximize the ability of a pressure wave created in a fluid to fracture calcium in an artery. For example, certain embodiments can be configured to generate a first electromagnetic energy emission that generates a vapor bubble in a fluid (e.g. ICG), where the vapor bubble initially expands and then collapses. Particular embodiments can be configured to also generate a second electromagnetic energy emission that is emitted at approximately the same time as the bubble generated from the first electromagnetic energy emission collapses. By timing the emission of the second electromagnetic energy pulse to occur when the vapor bubble from the first electromagnetic energy emission is collapsing, a larger pressure pulse can be created and the ability to fracture calcium in an artery or other environment can be enhanced.

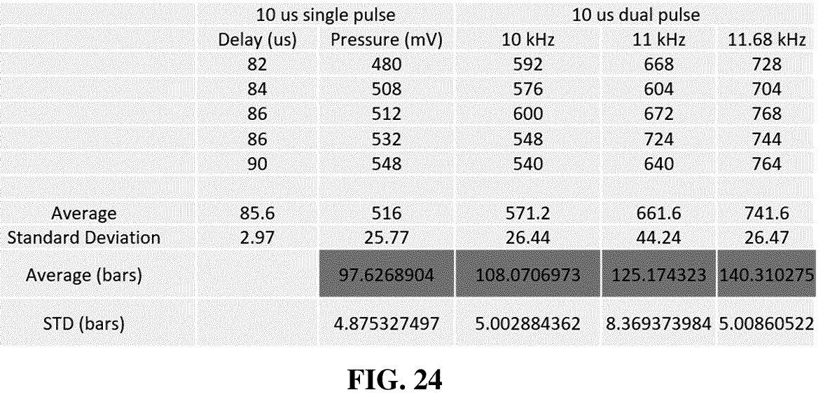

Referring now to FIGS. 24-29, data was collected from embodiments emitting a dual pulse of electromagnetic energy and compared to a similar embodiment emitting a single pulse of electromagnetic energy. Results were recorded for electromagnetic energy pulse duration of 10 μs, 20 μs, and 50 μs from a NLight© 1500-watt fiber laser at 10 volts and 793 nm wavelength. Data was collected with a pressure sensor approximately 13.73 mm from the fiber tip in a chamber pressurized to 4 bars filled with a 50/50 mixture of ICG (5 mg/mL) and Visipaque™ solution.

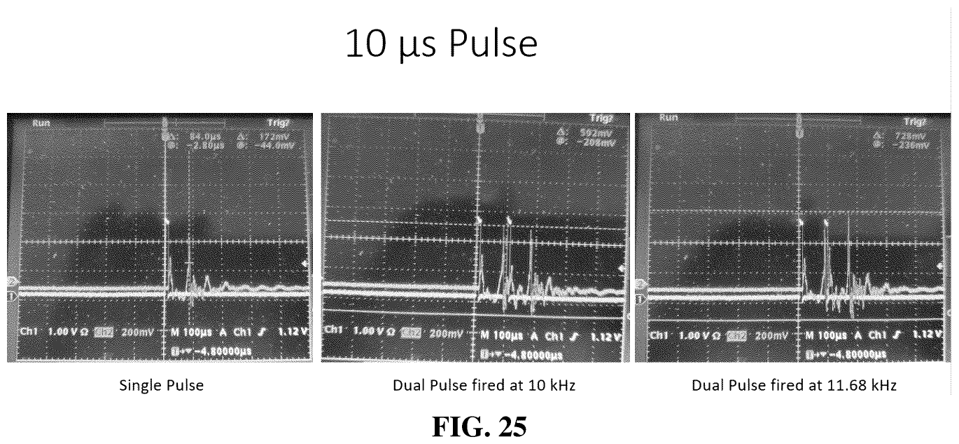

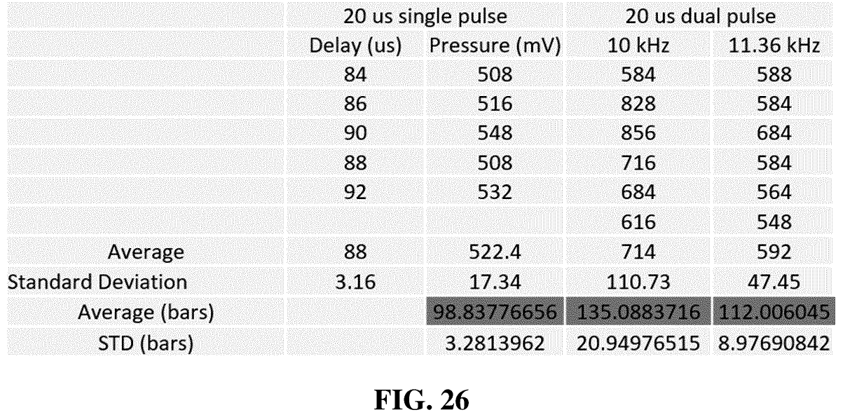

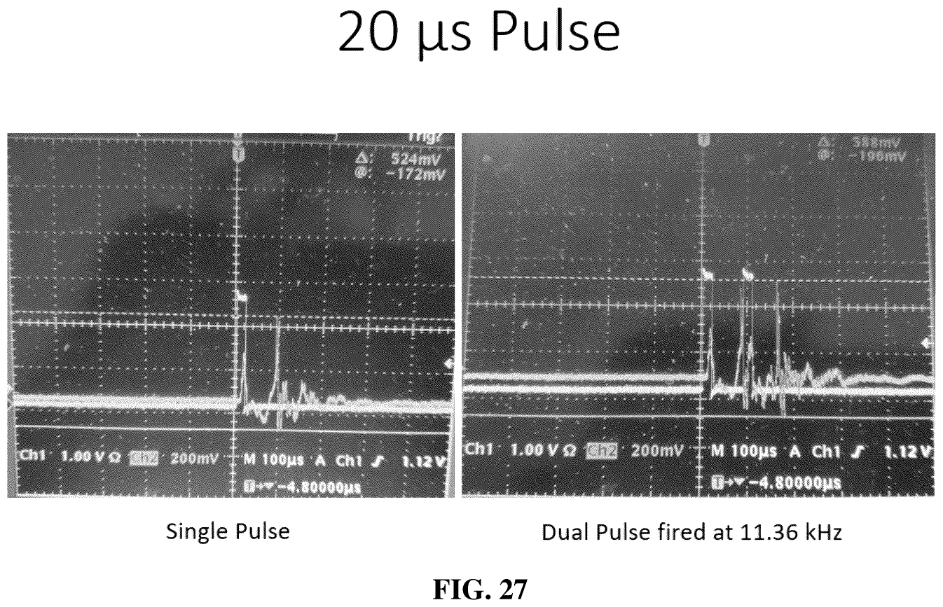

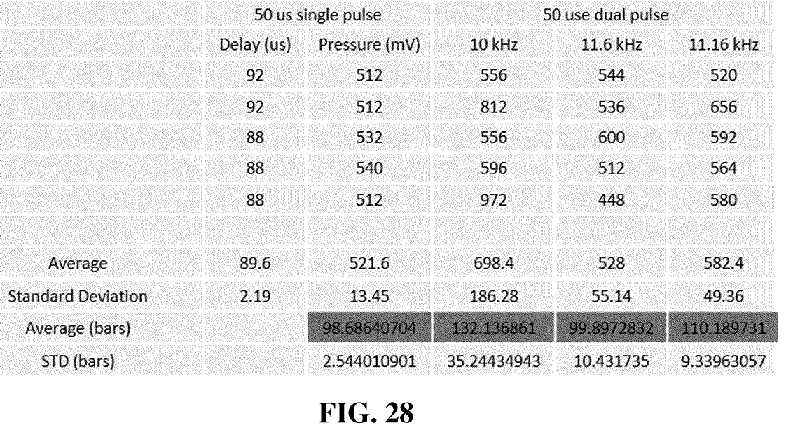

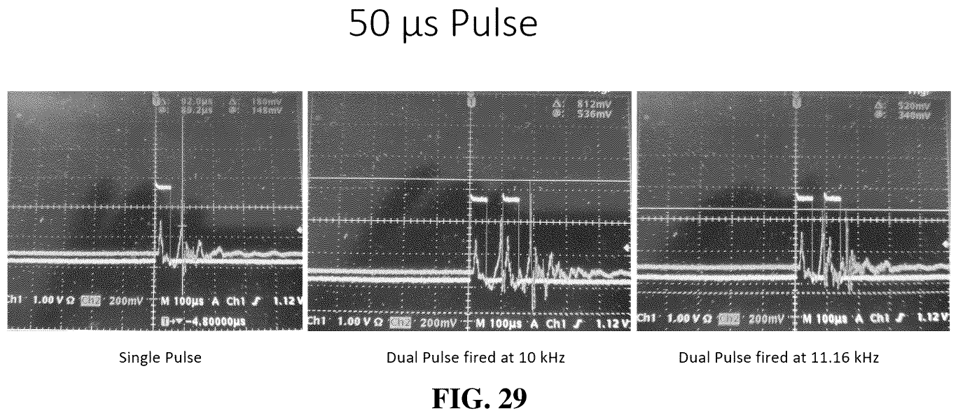

The data for the single pulse was collected initially to determine the delay between the laser pulse and the vapor bubble collapse. The single pulse data was collected five times to determine an average delay between the laser pulse and the vapor bubble collapse and the amplitude of the shockwave pressure. The dual pulse data was collected by firing a second laser pulse at the average time delay observed in the single pulse between the laser pulse and the vapor bubble collapse. The data recorded for the 10 μs laser pulse is shown in FIGS. 24-25, while the 20 μs laser pulse data is shown in FIGS. 26-27 and the 50 μs laser pulse data is shown in FIGS. 28-29. As noted in each of the charts, the average pressure recorded for the dual pulse embodiments was greater than that generated in the single pulse embodiments. The pressure data was recorded in millivolts from the pressure transducer and converted to bars in the average calculation. Pressure data for the dual pulse embodiments was collected at 10 kHz, 11 kHz and 11.68 kHz for the 10 μs embodiment, while data for the 20 μs embodiment was collected at 10 kHz and 11.36 kHz, and collected at 10 kHz, 11.6 kHz and 11.16 kHz for the 50 μs embodiment. The most significant difference was noted in the 10 μs laser pulse embodiment at 11.68 kHz, which provided an average pressure of 140.31 bars, as compared to 97.63 bars for the single pulse embodiment at 10 μs.

Particular embodiments of the present disclosure may also comprise a plurality of optical fibers, where each optical fiber is coupled to a separate diode laser. Such embodiments can provide increased flexibility with the operational parameters of the laser light emissions from the diode lasers. For example, the use of multiple separate optical fibers each coupled to an individual diode laser can allow a user to have increased spatio-temporal control by emitting light from the separate diode laser/optical fiber units in a manner that may not be possible with a single laser (or multiple lasers) coupled to a single optical fiber.

Several considerations are made in the configuration of a diode laser IVL catheter incorporating multiple optical fibers. A diode laser emitter provides a specified radiance (W/(sr area)) or Watts per unit Etendue. A laser IVL catheter specification requires a number of emitters, and each emitter in a laser IVL catheter needs to provide some minimum radiant power density (Watts/Area) to generate a shockwave. For example, for 5 mg/ml ICG a typical minimum radiant power density of approximately 2 kW/mm² is needed for shockwave generation. One challenge with laser IVL catheter design centers on the distribution of source radiance (W/Etendue) provided by diode laser emitters into catheter emitters. The optical etendue (capacity to carry light) of a fiber is proportional to the product of the core-area and solid angle (NA²).

IVL catheter design considerations include compatibility with existing guidewire (e.g. 0.014″ wire/350 μm) and minimizing the overall catheter diameter. For a laser IVL catheter, the diameter of each optical fiber contributes to the overall diameter of the laser IVL catheter. Accordingly, the use of small core diameter/high numerical aperture (NA) optical fibers provides a number of important advantages. For example, the small core diameter allows satisfying the overall design diameter constraint of the laser IVL catheter. In addition, fibers with a small core diameter provide increased radiant exitance (W/Area) at the fiber tip. Furthermore, fibers with high NA's increase the etendue of the fiber, and for a given diode laser emitter allow more efficient coupling of diode laser radiant emission into the fiber and allow coupling of more wattage into each fiber.

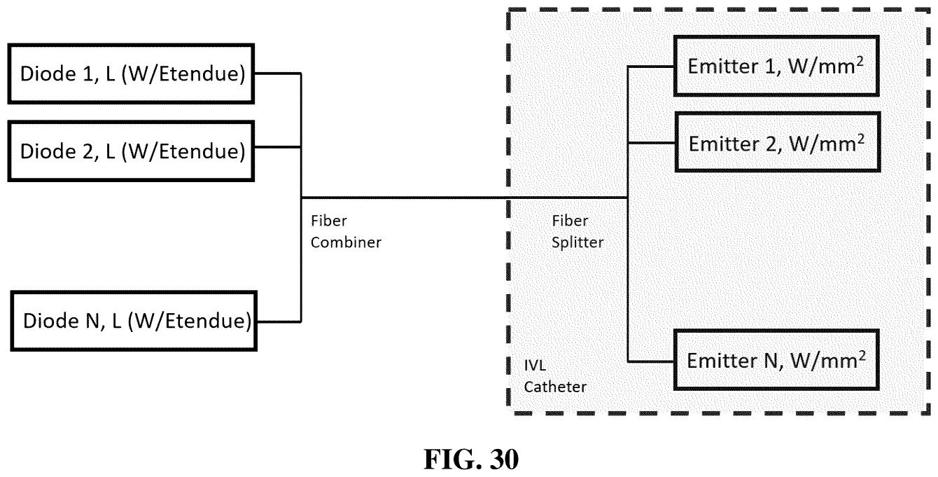

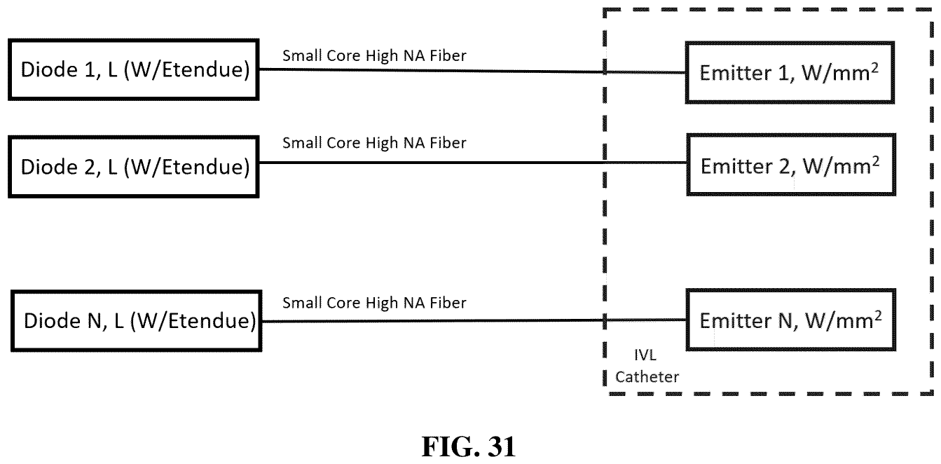

The use of a separate optical fiber for each diode laser can also reduce or eliminate the need for passive splitters/combiners (e.g. used to split or combine light paths from one or more laser sources). A block diagram of a system utilizing multiple diode lasers combined into a single fiber and then split into different fibers for each emitter is shown in FIG. 30. Passive combiner/splitters can add cost and complexity to the system design, and configurations that do not use either a combiner/splitter can provide systems that are simpler, have lower loss, and are more cost effective. With the use of passive combiner/splitters, all emitters coupled to a laser source emit radiation at the same time, and triggered emission from individual emitters is not possible. Accordingly, the use of small core high NA fibers allows direct coupling of individual laser diodes to each emitter, and the use of small core diameter high etendue fibers allows efficient and fiber specific generation of shock waves while maintaining a small diameter laser IVL catheter. A block diagram of a system using a separate small core high NA fiber coupled to each diode and emitter is shown in FIG. 31. In certain embodiments, the small core high NA fibers can be biocompatible Optran® Ultra WFGE doped Si/Si fiber, with glass/glass/polyamide configuration for the core/clad/coating having a diameter of 50/60/70 m.

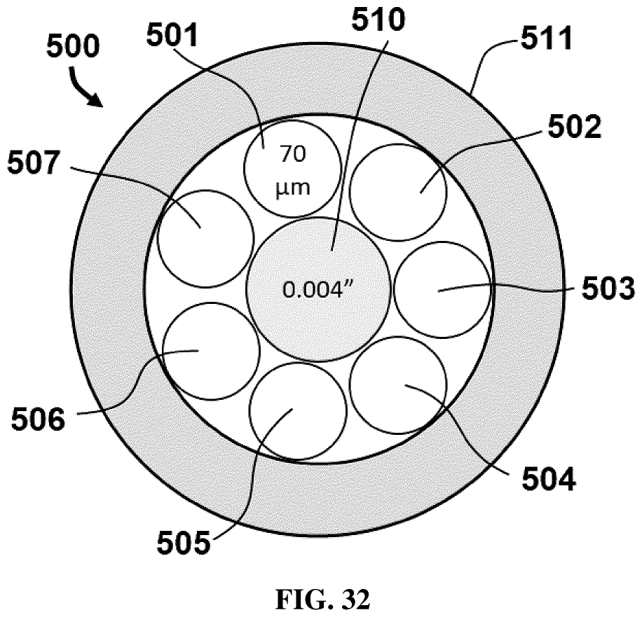

Referring now to FIG. 32, an end section view of one embodiment of an IVL catheter 500 is shown comprising a plurality of optical fibers 501-507 distributed around a central guide wire 510. Optical fibers 501-507 and guide wire 510 are contained within an outer sheath or coil 511. In specific embodiments optical fibers 501-507 can have an outer diameter of 70 m, while guide wire 510 may be approximately 0.004 inches in diameter and formed from stainless steel (e.g. 316, 304 or 302 stainless steel). In certain embodiments outer coil 511 may be approximately 0.002 inches thick with an overall diameter of approximately 0.0140 inches and can be configured to transmit torque to catheter 500. It is understood that the dimensions of components in this embodiment are merely exemplary, and other embodiments of the present disclosure may comprise similar components with different dimensions.

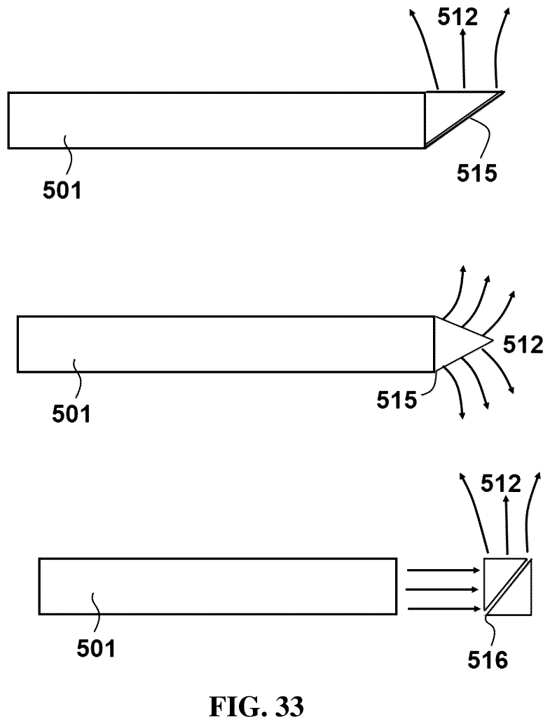

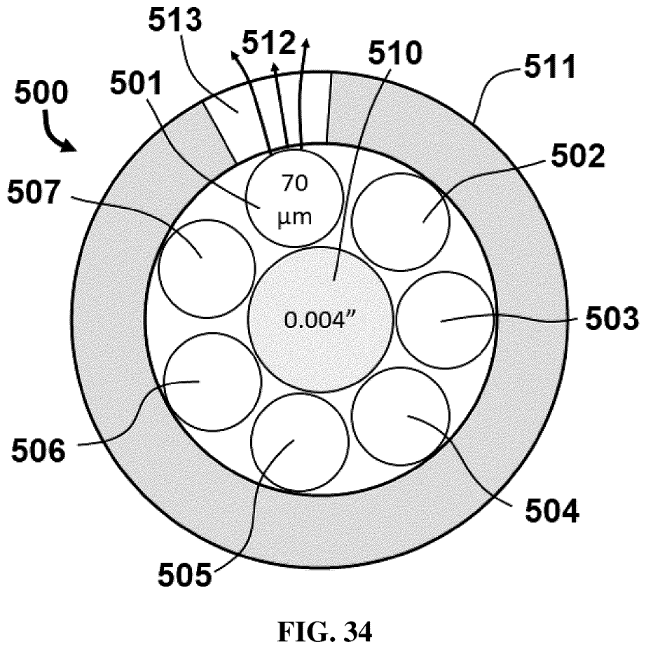

As shown in FIGS. 33-34, certain embodiments may also be configured to redirect laser light emissions 512 through one or apertures 513 in outer coil 511. In certain embodiments, an optical fiber (e.g. optical fiber 501) may comprise a distal end 515 that is conical, angled, tapered (or otherwise configured) to re-direct laser light emissions 512 via internal reflection. In certain embodiments, a conical distal end 515 can be configured to provide laser light emissions 512 that generate symmetric vapor bubbles when emitted in a fluid. The generation of symmetric vapor bubbles can maximize the amount of energy per vapor bubble volume transferred to coronary calcium during the subsequent collapse of the vapor bubble. In other embodiments, optical fiber 501 may direct laser light emissions 512 into a graded index (GRIN) lens 516 configured to direct laser light emissions 512 through aperture 513 (shown in FIG. 33).

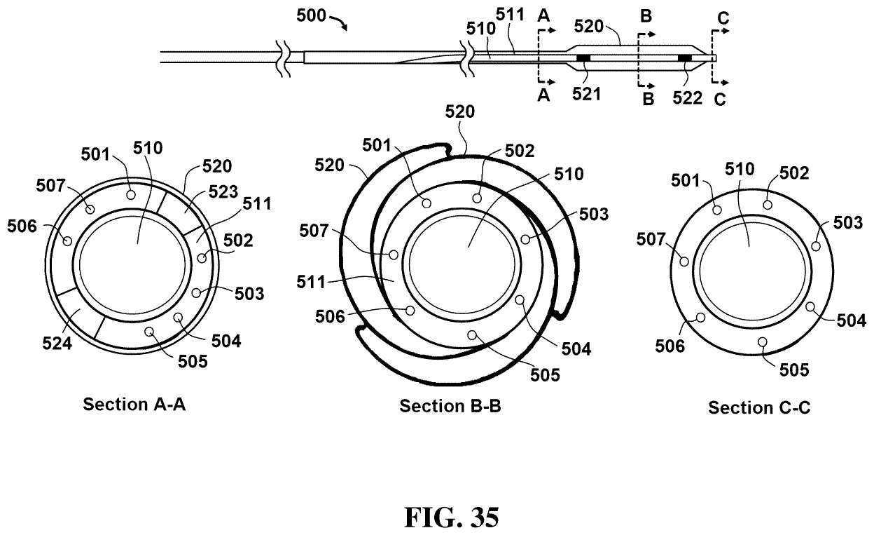

Referring now to FIG. 35, certain embodiments of the present disclosure may comprise configurations in which optical fibers are located within an expandable member (e.g. a balloon catheter). In FIG. 35, schematic section views are shown at section lines A-A, B-B and C-C of IVL catheter 500. In this embodiment, IVL catheter 500 comprises optical fibers 501-507 contained within expandable member 520. In the embodiment shown, expandable member 520 is configured as a catheter balloon that can fold around central guide wire 510 and outer coil 511 when expandable member is in a deflated condition. This configuration can reduce the overall diameter of expandable member 520 and allow expandable member 520 to be more easily placed in the desired location within an artery or other lumen. The location of expandable member 520 can be verified via radiopaque markers 521 and 522 before expandable member 520 is expanded (e.g. inflated via fluid delivered by a fluid delivery channels 523 and 524 shown in Section A-A view).

In certain embodiments, optical fibers 501-507 can be configured to direct laser light emissions through aperture in outer coil 511 as described in previous embodiments (e.g. through an aperture via a GRIN lens or the configuration of the optical fiber as shown in FIGS. 33-34). In particular embodiments expandable member 520 can be tapered from approximately 0.55 mm to approximately 0.75 mm, or from approximately 0.65 mm to approximately 0.8 mm. As previously discussed, multiple optical fibers 501-507 provide for increased spatio-temporal control of the laser light emission. This can allow a user to sequentially time the emission from one fiber to coincide with the collapse of a vapor bubble generated by a laser light emission previously emitted from another fiber (or the same fiber the laser light source is capable of such emission). In addition, by distributing optical fibers circumferentially around a lumen into which the IVL catheter is inserted, a user can direct a specific fiber to provide laser light emissions in a desired radial direction to target specific locations of interest.

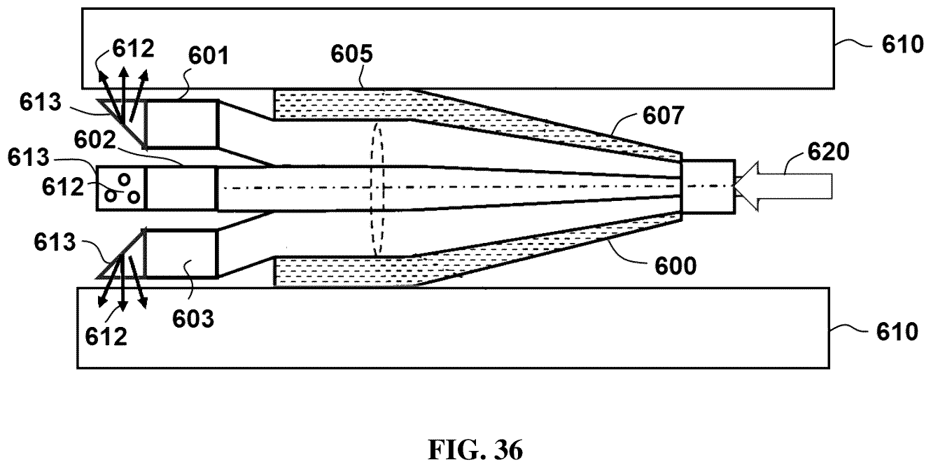

Certain embodiments of the present disclosure may comprise a tapered fiber coupler configured for use in an IVL catheter. Referring now to FIG. 36, a partial section view of an embodiment of a tapered fiber coupler 600 is located within a proximal end (e.g. the end proximal to the IVL catheter operator) of a sheath 610 for an optical guidewire. In certain embodiments sheath 610 may be a steel sheath and tapered fiber coupler 600 may include a housing comprising a glass tubular member 605 with a tapered region 607. In the embodiment shown light 620 enters tapered fiber coupler 600 and is directed to optical output fibers 601-603. In the partial section view shown in FIG. 36 three output fibers are shown in an embodiment comprising a total of seven output fibers. It is understood that other embodiments may comprise any number of output fibers within optical guidewire sheath 610. Each of the optical output fibers 601-603 comprises an emitter 613 (e.g. a side-firing reflector) configured to redirect laser light emissions 612 in an outwardly radial direction or other direction as desired.

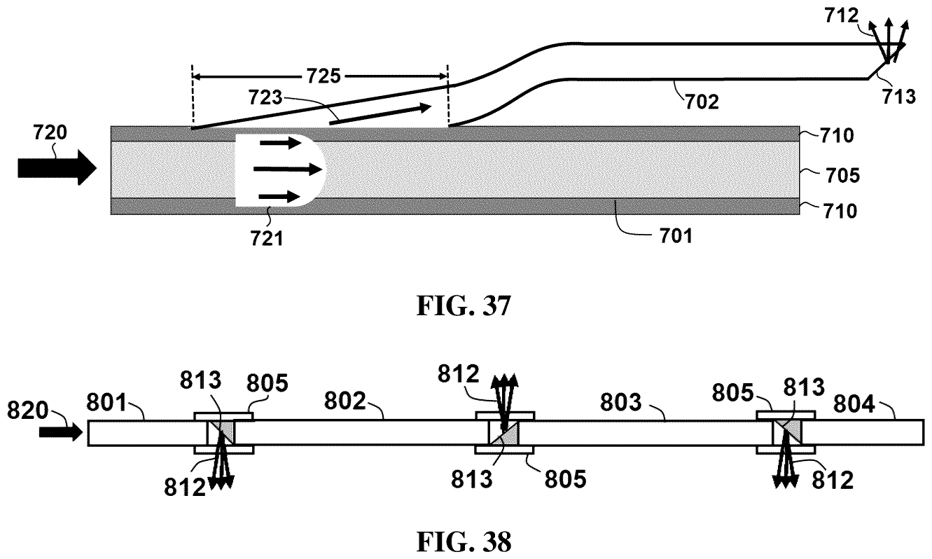

Particular embodiments of the present disclosure may also comprise one or more side-coupled fibers in which an evanescent field in a primary fiber couples into one or more emitter fibers. Referring now to FIG. 37, a primary fiber 701 comprising a core 705 and a cladding 710 is coupled with an emitter fiber 702 via a side-coupling region 725. Side-coupling region 725 can be formed via a fused biconical taper (FBT) process or other suitable methods. Light 721 propagates through primary fiber 701 via cladding 710 and via core 705. A portion of light 721 propagating via cladding 710 is directed to emitter fiber 702 as light 723 via side-coupling region 725. The fraction of light 721 that is transmitted to emitter fiber 702 as light 723 can be controlled by specifying the desired surface area within side-coupling region 725. For example, if more light 723 is desired, a configuration with a larger surface area in side-coupling region 725 can be specified. If less light 723 is desired, a configuration with a smaller surface area in side-coupling region 725 can be specified. Light 723 propagates through emitter fiber 702 to an emitter 713 (e.g. a side-firing reflector) configured to redirect laser light emissions 712 in an outwardly radial direction or other direction as desired. While the embodiment shown in FIG. 37 illustrates one side-coupled fiber 702, it is understood that other embodiments may comprise additional side-coupled fibers.

Specific embodiments of the present disclosure may also comprise optical fibers with inline reflectors comprising dielectric films. Referring now to FIG. 38, a plurality of optical fibers 801-804 are coupled via coupling elements 805 In the embodiment shown, optical fibers 801-804 comprise an emitter 813 configured as a polished end that is tapered or angled (e.g. at 45 degrees in the embodiment shown) and coated with a dielectric reflector. Light 820 propagates via fibers 801-804 and is redirected outwardly in a radial direction via emitters 813 as laser light emissions 812. In certain embodiments coupling elements 805 can be configured as transparent or translucent sleeves that function as lens elements configured to focus laser light emissions 812.

All of the apparatus, systems and/or methods disclosed and claimed herein can be made and executed without undue experimentation in light of the present disclosure. While the apparatus, systems and methods of this invention have been described in terms of particular embodiments, it will be apparent to those of skill in the art that variations may be applied to the devices, systems and/or methods in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

The contents of the following references are incorporated by reference herein:
1. Rocha-Singh et al, Peripheral arterial calcification: prevalence, mechanism, detection, and clinical implications, Catheter Cardiovasc Intervention, 2014
2. Ali et al, Optical Coherence Tomography Characterization of Coronary Lithoplasty for Treatment of Calcified Lesions, JACC Imaging, 2017
3. Warisawa et al, Successful Disruption of Massive Calcified Nodules Using Novel Shockwave Intravascular Lithotripsy, Circ J, 2020
4. Brinton et al, Feasibility of Shockwave Coronary Intravascular Lithotripsy for the Treatment of Calcified Coronary Stenoses, Circ J, 2019
U.S. Pat. No. 8,728,091
U.S. Pat. No. 8,747,416
U.S. Pat. No. 8,888,788
U.S. Pat. No. 8,956,371
U.S. Pat. No. 8,956,374
U.S. Pat. No. 9,005,216
U.S. Pat. No. 9,011,462
U.S. Pat. No. 9,011,463
U.S. Pat. No. 9,072,534
U.S. Pat. No. 9,138,249
U.S. Pat. No. 9,333,000
U.S. Pat. No. 9,433,428
U.S. Pat. No. 9,642,673
U.S. Pat. No. 10,206,698
U.S. Pat. No. 10,039,561
U.S. Pat. No. 10,159,505
U.S. Pat. No. 10,517,621
U.S. Pat. No. 10,682,178
U.S. Pat. No. 10,702,293
U.S. Pat. No. 10,709,462
U.S. Patent Publication 2022/0183756
Intl. Pat. Pub. WO 2022/125919

1. An apparatus comprising:
a diode laser light source; and
an optical fiber, wherein the optical fiber comprises:
a polymer optical core;
a cladding surrounding the polymer optical core; and
a laser light emission element.

2. The apparatus of claim 1 wherein the laser light emission element is a first laser light emission element in a plurality of laser light emission elements.

3. The apparatus of claim 2 wherein each of the plurality of laser light emission element is configured to emit light at an equivalent wavelength range.

4. The apparatus of claim 2 wherein each of the plurality of laser light emission elements is configured to emit light at equivalent power.

5. The apparatus of claim 2 wherein:
a first laser light emission element of the plurality of laser light emission elements is configured to emit light at a first wavelength range;
a second laser light emission element of the plurality of laser light emission elements is configured to emit light at a second wavelength range; and
the first wavelength range is different than the second wavelength range.

6. The apparatus of claim 2 wherein an optical grating within the optical fiber comprises the plurality of laser light emission elements.

7. The apparatus of any one of claims 2-6 wherein the plurality of laser light emission elements emits light radially from the optical fiber.

8. The apparatus of any one of claims 2-7 wherein the plurality of laser light emission elements is configured as a line of scattering centers along the polymer optical core of the optical fiber.

9. The apparatus of any one of claims 2-7 wherein the plurality of laser light emission elements is configured as scattering centers located at positions offset from the polymer optical core and placed at equivalent angles near the cladding.

10. The apparatus of any one of claims 2-7 wherein the plurality of laser light emission elements is configured as one or more photonic crystal lattices comprising a plurality of scatting centers in the polymer optical core.

11. The apparatus of claim 7 wherein the plurality of laser light emission elements comprises N number of laser light emission elements, and wherein laser light emission elements are positioned radially around the optical fiber such that there are 360/N degrees between each laser light emission element in the plurality of laser light emission element.

12. The apparatus of claim 7 wherein the plurality of laser light emission elements emits light radially 360 degrees around the optical fiber.

13. The apparatus of any one of claims 1-12 wherein the diode laser light source is configured to emit laser light at a wavelength between approximately 690 nanometers (nm) and 900 nm.

14. The apparatus of any one of claims 1-13 wherein the diode laser light source can provide a pulse of light between 50 nanoseconds and 150 microseconds.

15. The apparatus of any one of claims 1-14 wherein radiant power propagating in the optical fiber is between 100 watts (W) and 100 kilowatts (kW).

16. The apparatus of any one of claims 1-15 wherein the polymer optical core comprises poly(methyl methacrylate) (PMMA), polydimethylsiloxane (PDMS), polyacrylamide (PAM) or a transparent amorphous fluoropolymer.

17. The apparatus of any one of claims 1-15 wherein the polymer optical core comprises a transparent thermoplastic.

18. The apparatus of claim 17 wherein the transparent thermoplastic is poly(methyl methacrylate).

19. The apparatus of any one of claims 1-15 wherein the polymer optical core comprises a silicon-based organic polymer.

20. The apparatus of claim 19 wherein the silicon-based organic polymer is polydimethylsiloxane.

21. The apparatus of any one of claims 1-15 wherein the polymer optical core comprises a transparent amorphous fluoropolymer.

22. The apparatus of any one of claims 1-15 wherein the polymer optical core comprises a synthetic polymer.

23. The apparatus of any one of claims 1-19 further comprising an expandable member.

24. The apparatus of claim 23 wherein the expandable member comprises a lumen configured to receive the optical fiber.

25. The apparatus of claim 23 or claim 24 wherein the expandable member contains a fluid.

26. The apparatus of claim 25 wherein the fluid surrounds the optical fiber and wherein the fluid absorbs light emitted by the diode laser light source.

27. The apparatus of claim 25 wherein the fluid comprises indocyanine green (ICG).

28. The apparatus of claim 27 wherein the fluid comprises a solvent.

29. The apparatus of claim 28 wherein the concentration of the ICG to the solvent is between 5 milligrams/milliliter (mg/ml) and 25 mg/ml.

30. The apparatus of claim 28 or 29 wherein the solvent comprises water, saline or dextrose.

31. The apparatus of any one of claims 1-30 further comprising a control system configured to control an operational parameter of the diode laser light source.

32. The apparatus of claim 31 wherein the operational parameter is a pulse duration, a wavelength frequency, multiple varying wavelength frequencies, or a wavelength amplitude of the diode laser light source.

33. The apparatus of any one of claims 25-31 wherein the control system is configured to provide a first laser light emission and a second laser light emission from the diode laser light source.

34. The apparatus of claim 33 wherein the first laser light emission is configured to generate a bubble in the fluid in the expandable member.

35. The apparatus of claim 34 wherein the control system is configured to provide the second laser light emission from the diode laser light source when the bubble in the fluid in the expandable member collapses.

36. The apparatus of any one of claims 1-32 wherein the optical fiber comprises an imaging element.

37. The apparatus of claim 36 wherein the imaging element is configured to provide intravascular ultrasound (IVUS) or optical coherence tomography (OCT) imaging data.

38. The apparatus of any one of claims 1-36 wherein:
the diode laser light source is a first diode laser light source in a plurality of diode laser light sources; and
the optical fiber is a first optical fiber in a plurality of optical fibers.

39. The apparatus of claim 38 wherein each diode laser light source in the plurality of diode laser light sources is coupled to a separate optical fiber in the plurality of optical fibers.

40. The apparatus of claim 38 wherein an optical fiber in the plurality of optical fibers comprises a conical distal end.

41. The apparatus of any one of claims 38-40 wherein the plurality of optical fibers are coupled via a tapered fiber coupler.

42. The apparatus of any one of claims 38-40 wherein the plurality of optical fibers are coupled via a side-coupling region.

43. The apparatus of any one of claims 38-40 wherein the plurality of optical fibers are coupled via sleeve coupling elements and at least one of the plurality of optical fibers comprises an angled polished end coated with a dielectric reflector.

44. An apparatus comprising:
a diode laser light source; and
an optical fiber, wherein the optical fiber comprises:
an optical core;
a cladding surrounding the polymer optical core; and
a plurality of laser light emission elements, wherein the laser light emission elements are configured as emission centers in the optical core.

45. The apparatus of claim 44 wherein the plurality of laser light emission elements is configured as a line of scattering centers along the optical core of the optical fiber.

46. The apparatus of claim 44 wherein the plurality of laser light emission elements is configured as scattering centers located at positions offset from the optical core and placed at equivalent angles near the cladding.

47. The apparatus of claim 44 wherein the plurality of laser light emission elements is configured as one or more photonic crystal lattices comprising a plurality of scatting centers in the optical core.

48. The apparatus of any one of claims 44-47 wherein the optical core is a polymer optical core.

49. The apparatus of any one of claims 44-47 wherein the optical core is a glass optical core.

50. The apparatus of any one of claims 44-49 wherein:
the diode laser light source is a first diode laser light source in a plurality of diode laser light sources; and
the optical fiber is a first optical fiber in a plurality of optical fibers.

51. The apparatus of claim 44-50 wherein each diode laser light source in the plurality of diode laser light sources is coupled to a separate optical fiber in the plurality of optical fibers.

52. The apparatus of claim 44-51 wherein an optical fiber in the plurality of optical fibers comprises a conical distal end.

53. The apparatus of any one of claims 50-52 wherein the plurality of optical fibers are coupled via a tapered fiber coupler.

54. The apparatus of any one of claims 50-52 wherein the plurality of optical fibers are coupled via a side-coupling region.

55. The apparatus of any one of claims 50-52 wherein the plurality of optical fibers are coupled via sleeve coupling elements and at least one of the plurality of optical fibers comprises an angled polished end coated with a dielectric reflector.

56. A method of fracturing calcium in an artery, the method comprising:
inserting an optical fiber into an artery, wherein:
the optical fiber is coupled to a diode laser light source; and
the optical fiber comprises:
a polymer optical core;
a cladding surrounding the polymer optical core; and
a laser light emission element;
inserting an expandable member into the artery;
expanding the expandable member via a fluid in the expandable member;
emitting electromagnetic energy from the laser light emission element, wherein the electromagnetic energy generates a pressure wave in the fluid contained within the expandable member; and
fracturing the calcium in the artery via the pressure wave in the fluid.

57. The method of claim 56 wherein the laser light emission element is a first laser light emission element in a plurality of laser light emission elements.

58. The method of claim 57 wherein each of the plurality of laser light emission elements is configured to emit light at an equivalent wavelength range.

59. The method of claim 57 wherein each of the plurality of laser light emission elements is configured to emit light at equivalent power.

60. The method of claim 57 wherein:
a first laser light emission element of the plurality of laser light emission elements is configured to emit light at a first wavelength range;
a second laser light emission element of the plurality of laser light emission elements is configured to emit light at a second wavelength range; and
the first wavelength range is different than the second wavelength range.

61. The method of claim 57 wherein a grating structure within the optical fiber comprises an element of each laser light emission element.

62. The method of any one of claims 57-61 wherein the plurality of laser light emission elements emits light radially from the optical fiber.

63. The method of claim 62 wherein the plurality of laser light emission elements comprises N number of laser light emission elements, and wherein laser light emission elements are positioned radially around the optical fiber such that there are 360/N degrees between each laser light emission element in the plurality of laser light emission element.

64. The method of claim 62 wherein the plurality of laser light emission elements emits light radially 360 degrees around the optical fiber.

65. The method of any one of claims 56-64 wherein the diode laser light source is configured to emit laser light at a wavelength between approximately 690 nanometers (nm) and 900 nm.

66. The method of any one of claims 56-65 wherein the diode laser light source can provide a pulse of light between 50 nanoseconds and 150 microseconds.

67. The method of any one of claims 56-66 wherein radiant power propagating in the optical fiber is between 100 watts (W) and 100 kilowatts (kW).

68. The method of any one of claims 56-67 wherein the polymer optical core comprises a synthetic polymer.

69. The method of any one of claims 56-67 wherein the polymer optical core comprises poly(methyl methacrylate) (PMMA), polydimethylsiloxane (PDMS), polyacrylamide (PAM) or a transparent amorphous fluoropolymer.

70. The method of any one of claims 56-67 wherein the polymer optical core comprises a transparent thermoplastic.

71. The method of claim 70 wherein the transparent thermoplastic is poly(methyl methacrylate).

72. The method of any one of claims 56-67 wherein the polymer optical core comprises a silicon-based organic polymer.

73. The method of claim 72 wherein the silicon-based organic polymer is polydimethylsiloxane.

74. The method of any one of claims 56-67 wherein the polymer optical core comprises a transparent amorphous fluoropolymer.

75. The method of any one of claims 56-74 wherein the fluid comprises indocyanine green (ICG).

76. The method of claim 75 wherein the fluid comprises a solvent.

77. The method of claim 76 wherein the concentration of the ICG to the solvent is between 5 milligrams/milliliter (mg/ml) and 25 mg/ml.

78. The method of claim 76 or 77 wherein the solvent comprises water, saline or dextrose.

79. The method of any one of claims 56-78, wherein:
the expandable member comprises a lumen; and
the optical fiber extends through the lumen of the expandable member.

80. The method of any one of claims 56-79 wherein the optical fiber comprises an imaging element.

81. The method of claim 80 wherein the imaging element is configured to provide intravascular ultrasound (IVUS) or optical coherence tomography (OCT) imaging.

82. The method of claim 80 or 81 wherein the imaging element provides imaging data while:
inserting the optical fiber into the artery;
inserting the expandable member into the artery;
expanding the expandable member via a fluid in the expandable member;
emitting electromagnetic energy from the laser light emission element; or
fracturing the calcium in the artery via the pressure wave in the fluid.

83. The method of any one of claims 80-82 wherein the imaging element provides imaging data after fracturing the calcium in the artery via the pressure wave in the fluid.

84. The method of any one of claims 56-82 wherein:
the electromagnetic energy is a first pulse of electromagnetic energy that generates a bubble in the fluid in the expandable member; and
the bubble collapses after the bubble is generated.

85. The method of claim 84 further comprising emitting a second pulse of electromagnetic energy, wherein the second pulse of electromagnetic energy is emitted after the first pulse of electromagnetic energy.

86. The method of claim 85 wherein the second pulse is emitted approximately when the bubble collapses.

87. The method of any one of claims 56-86 wherein:
the diode laser light source is a first diode laser light source in a plurality of diode laser light sources; and
the optical fiber is a first optical fiber in a plurality of optical fibers.

88. The method of any one of claims 56-87 wherein each diode laser light source in the plurality of diode laser light sources is coupled to a separate optical fiber in the plurality of optical fibers.

89. The method of any one of claims 56-88 wherein an optical fiber in the plurality of optical fibers comprises a conical distal end.
